SEQUENCE LISTING (7 sequences)

SEQ ID NO: 1 - DNA, 14 bp, Artificial Sequence
  Primer
gcggacttcctcgg

SEQ ID NO: 2 - DNA, 13 bp, Artificial Sequence
  Primer
tcgcgagcttcct

SEQ ID NO: 3 - DNA, 20 bp, Artificial Sequence
  Oligonucleotide WJ31
ctcagagggccgctgcgccc

SEQ ID NO: 4 - DNA, 21 bp, Artificial Sequence
  Oligonucleotide WJ32
ccagcgcagctagcggggcag

SEQ ID NO: 5 - DNA, 27 bp, Artificial Sequence
  Oligonucleotide WJ
acaccagcgcagctcgaggggcagccg

SEQ ID NO: 6 - DNA, 40 bp, Artificial Sequence
  Oligonucleotide WJ47
gcgctacgtactcgaggccccagccagccccgacggcgcc

SEQ ID NO: 7 - DNA, 41 bp, Artificial Sequence
  Oligonucleotide WJ48
cgcgtacgtatgatcatcagcccgagcactgctgagacgac

(19) United States
(12) Patent Application Publication
(10) Pub. No.: US 2002/0119095 A1
(43) Pub. Date: Aug. 29, 2002

(54) Chemotherapeutic agents conjugated to p97 and their methods of use in treating neurological tumours

(76) Inventors: Reinhard Gabathuler, Vancouver (CA); Gerrassimos Kolaitis, Vancouver (CA); Robert Charles Brooks, Burnaby (CA); Qingqi Chen, Vancouver (CA); Delara M. Karkan, Vancouver (CA); Gavin D. Arthur, Vancouver (CA); Jean Paul St. Pierre, Beaconsfield (CA); Wilfred Jeffries, South Surrey (CA); Timothy Z. Vitalis, Vancouver (CA)

Correspondence Address: BERESKIN AND PARR, SCOTIA PLAZA, 40 KING STREET WEST-SUITE 4000 BOX 401, TORONTO, ON, M5H 3Y2, CA

(21) Appl. No.: 09/931,184

(22) Filed: Aug. 17, 2001

Related U.S. Application Data
(63) Continuation-in-part of application No. 09/285,040, filed on Apr. 1, 1999.
(62) Division of application No. 08/520,933, filed on Aug. 31, 1995.
(63) Continuation-in-part of application No. 08/367,224, filed on Mar. 30, 1995, now abandoned.
(63) Continuation-in-part of application No. 07/912,291, filed on Jul. 10, 1992, now abandoned.
(60) Provisional application No. 60/226,254, filed on Aug. 17, 2000.

(51) Int. Cl. A61K 51/00; A61K 39/395; C07K 16/46

(52) U.S. Cl. 424/1.49; 530/391.1; 424/178.1

(57) ABSTRACT

The present invention provides pharmaceutical compositions of chemotherapeutic agents that demonstrate therapeutic efficacy against brain tumours and other neoplasia localized in or around the brain. In certain aspects, the pharmaceutical compositions comprise a chemotherapeutic agent conjugated to p97; and a pharmaceutically acceptable carrier therefor. Preferred chemotherapeutic agents include, but are not limited to, adriamycin, cisplatin, and paclitaxel.

This application is a continuation-in-part of U.S. Ser. No. 09/285,040 filed Apr. 1, 1999 which is a divisional of U.S. Ser. No. 08/520,933 filed Aug. 31, 1995 (now U.S. Pat. No. 5,981,194) which is a continuation-in-part of U.S. Ser. No. 08/367,224, filed Mar. 30, 1995 (now abandoned) which is a continuation-in-part of U.S. Ser. No. 07/912,291, filed Jul. 10, 192 (now abandoned). This application also claims the benefit under 35 USC 119(e) off U.S. Provisional Application No. 60/26,254 filed Aug. 17, 2000. All of the prior applications are incorporated herein by reference in their entirety.

### FIELD OF THE INVENTION

The present invention relates to drug delivery compositions for enhanced delivery of chemotherapeutic agents to tumours in or around the brain, and for reducing the systemic toxicity of chemotherapeutic agents used in treating tumours in and around the brain.

### BACKGROUND OF THE INVENTION

p97, also known as melanotransferrin (or Mt), is a human melaoma-associated protein antigen. It was one of the first cell surface markers to be associated with human skin cancer (see, Hellstrom, K. E. and Hellstrom, I., (1982) in Melanoma Antigens and Antibodies, Ed. Reisfield, R. and Ferrone, S., Plenum Press, New York, pp. 187-341). p97 is a monomeric membrane-associated protein with a molecular mass of 97,000 daltons (see, Brown, J. P. et al. J. Immunol. 127:539, 1981) and has been suggested as a melanoma specific marker (see, Estin, C. D. et al., Proc. Nat. Acad. Sci. USA, 85:1052-1056, 1988). In addition, it has been associated with the cell surface of melanomas and some other tumours and cell lines (see, Brown, J. P. et al., Proc. Nat. Acad. Sci. U.S.A. 78:539, 1981); p97 has also been found in certain fetal tissue (see, Woodbury, R. G. et al., Int. J. Cancer 27:145, 1981) and, more recently, on endothelial cells of the human liver (see, Sciot, R, et al., Liver 9:110, 1989). Homologs of p97 have now been identified in mouse, chicken, pig, and rabbit.

The primary structure of p97, deduced from its mRNA sequence, indicates that it belongs to a group of closely related iron binding proteins found in vertebrates (see, Rose, T. M. et al., Proc. Nat. Acad, Sci. U.S.A. 83:1261, 1986). This family includes serum transferrin, lactoferrin and avian egg white ovotransferrin. Human p97 and lactoferrin share 40% sequence homology (see, Baker, E. N. et al., Trends Biochem. Sci. 12:350, 1987), however in contrast to the other molecules of the transferrin family; p97 is the only one which is directly associated with the cell membrane. The deduced sequence of p97 has, in addition to a transferrin-like domain, a hydrophobic segment at its C terminal. This hydrophobic C terminus is cleaved post-translationally. A glycosyl phosphatidyl inositol is attached to p97 to generate the predominant membrane bound form of the mature molecule (see, Food et al. 1994, J. Biol. Chem. 269(4):3034-3040).

Published work on p97 has most recently focussed on its possible physiological roles as a diagnostic indicator of Alzheimer's disease, and a highly selective transporter of iron across the blood-brain barrier. (see, Kennard et al. 1996. Nat. Med. 2(11):1230-1235; Yamada et al. 1999. Brain Res. 845:1-5)

Brain tumour therapy and treatment continues to be a major challenge for physicians. Certain kinds of brain tumours are non-responsive to a wide variety of chemotherapeutic treatments used routinely against other tumour types. This effect may be attributed to the blood brain barrier that prevents certain compounds, and particularly strongly ionized agents such as quaternary amines, from entering the brain or the cerebro-spinal fluid from the circulation. No effective treatments have been established for glioblastoma multiforme or high-grade astrocytomas; and certain other brain tumours are amenable to radiation or surgery only.

It is an object of the instant invention to provide methods and compositions for treating brain tumours and other neoplasia in and around the brain, by employing a chemotherapeutic agent linked to p97.

### SUMMARY OF THE INVENTION

This invention now demonstrates that chemotherapeutic agents which are linked to p97, thus forming a p97-chemotherapeutic agent composition, are excellent vehicles for enhanced delivery of the chemotherapeutic agents to brain tumours and other neoplasia localized in or around the brain, and for improved treatment of such tumours and neoplasia.

In one embodiment, the present invention provides formulations of chemotherapeutic agents which demonstrate therapeutic efficacy against brain tumours and other neoplasia localized in or around the brain, but which chemotherapeutic agents, in the free form, demonstrate no therapeutic efficacy against such tumours and neoplasia. Preferred formulations comprise p97 linked to such chemotherapeutic agents. Preferred chemotherapeutic agents include, but are not limited to, adriamycin, cisplatin, and paclitaxel.

In another embodiment, the present invention provides a p97-chemotherapeutic agent with improved therapeutic efficacy against a brain tumour or other neoplasia located in or around the brain.

Preferred compositions have from about 1 to about 20 molecules of the chemotherapeutic agent linked to a single p97 molecule to form a p97-chemotherapeutic agent.

In a further embodiment, the present invention provides novel p97-chemotherapeutic agent conjugates along with modified forms of p97 and chemotherapeutic agents useful for preparing the conjugates of the invention.

In another embodiment, the present invention provides a method of treating a brain tumour or other neoplasia located in or around the brain comprising administering an effective amount of a composition comprising a chemotherapeutic agent conjugated to p97 to an animal in need thereof. The invention also provides a use of a composition comprising a chemotherapeutic agent conjugated to p97 to prepare a medicament to treat a brain tumour or other neoplasia located in or around the brain.

Other features and advantages of the present invention will become apparent from the following figures and detailed description. It should be understood, however, that the detailed description and the specific examples while indicating preferred embodiments of the invention are given by way of illustration only, since various changes and modification within the spirit and scope of the invention will become apparent to those skilled in the art from this detailed description.

### BRIEF DESCRIPTION OF THE DRAWINGS

FIG. 1 illustrates results of the effect of varying the ratio of activated ADR to p97 on the MSR of the resulting conjugate.

FIG. 2 illustrates tissue/serum ratio of p97-I¹²⁵ (Apo and holo) versus BSA-I¹²³ at 1 hour post-i.v. injection.

FIG. 3 illustrates relative % increase in uptake of p97-I¹²⁵ (Apo and holo) versus uptake of BSA-I¹²⁵ at 15 mins. after administration.

FIG. 4 illustrates relative % increase in uptake of p97-I¹²⁵ (Apo and holo) versus uptake of BSA-I¹²⁵ at 1 hour after administration.

FIG. 5 is a bar graph comparing the accumulation of ¹²⁵I-p97 and ¹²⁵BSA in the brain.

FIG. 6 is a bar graph comparing the accumulation of ¹²⁵I-p97 and ¹²⁵I-BSA in the brain, spinal cord and neurological tumour.

FIG. 7 illustrates comparison of tissue distribution of p97-ADR and free ADR at 1 hour after administration.

FIG. 8 illustrates comparison of uptake of p97-ADR and free ADR by heart tissue.

FIG. 9 illustrates survival of C6 Glioma intracranial tumour bearing mice in response to treatment by p97 ADR.

FIG. 10 is a graph showing the % survival of mice injected with IC C6 glioma and treated with PBS (control) and p97-ADR conjugates.

FIG. 11 illustrates survival of ADR-75-1 intracranial tumour bearing mice in response to treatment by p97-ADR and free ADR.

### DETAILED DESCRIPTION OF THE INVENTION

In certain aspects, the present invention provides compositions, and methods for using these compositions in treating brain tumours and other neoplasia in an around the brain, comprising p97 linked to a chemotherapeutic agent. Such tumours or neoplasia may be primary tumours or may be metastases. Preferred compositions have from about 1 to about 20 molecules of the chemotherapeutic agent linked to each p97 molecule.

### I. DEFINITIONS

“p97” is a micrometric protein with a molecular mass of 97,000 daltons that is also referred to as melanotransferrin. “p97” as used in the compositions of the invention, includes membrane bound p97 (i.e., p97 linked to GPI or other lipids), soluble p97, cleaved p97, analogs of p97 which are equivalents of p97 (having greater than 40% homology at the peptide sequence level, including allelic variants of p97), p97 from all species including human, mouse, chicken and/or rabbit p97, and derivatives, portions, or fragments thereof. p97 may be in the form of acidic or basic salts, or in neutral form. In addition, individual amino acid residues may be modified, such as by oxidation or reduction. Various substitutions, deletions, or additions may be made to the amino acid or DNA nucleic acid sequences, the net effect of which is to retain or improve upon the desired biological activity of p97. Due to code degeneracy, for example, there may be considerable variation in nucleotide sequences encoding the same amino acid sequence. As used herein, p97 also includes fragments of p97, including any portion of p97 or its biologically equivalent analogs that contain a sufficient portion of p97 to enable it to retain or improve upon the desired biological activities of p97. Further p97 also includes p97 and its analogs that have been modified to incorporate reactive groups for attaching to linker molecules and/or the chemotherapeutic agent(s).

“p97-chemotherapeutic agent” as used herein means a composition comprising p97 (including p97 fragments) conjugated to a chemotherapeutic agent. The conjugation may be direct or indirect (i.e., through an extended linker). Examples of general constructs of the compositions of the invention are as follows:

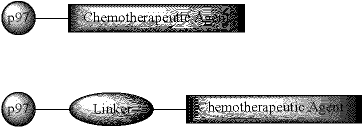

The “chemotherapeutic agent” is any chemical agent that can be used to treat a disease. Preferred chemotherapeutic agents for use in p97-chemotherapeutic agents of the invention include all drugs which may be useful for treating brain tumours or other neoplasia in or around the brain, either in the free form, or, if not so useful in the free form, then useful when linked to p97. Such chemotherapeutic agents include adriamycin (also known as doxorubicin), cisplatin, paclitaxel, camptothecin, 5-fluorouracil, analogs thereof, and other chemotherapeutic agents which demonstrate activity against tumours ex vivo and in vivo. Such chemotherapeutic agents also include alkylating agents, antimetabolites, natural products (such as vinca alkaloids, epidophyllotoxins, antibiotics, enzymes and biological response modifiers), topoisomerase inhibitors, microtubule inhibitors, spindle poisons, hormones and antagonists, and miscellaneous agents such as platinum coordination complexes, anthracendiones, substituted urcas, etc. those of skill in the art will know of other chemotherapeutic agents.

In certain aspects, the therapeutic agent and p97 are conjugated. As used herein, the term “conjugated” means that the therapeutic agent(s) and p97 are physically linked by, for example, covalent chemical bonds, physical forces such van der Waals or hydrophobic interactions, encapsulation, embedding, chelation, or combinations thereof. In a preferred embodiment, the therapeutic agent and p97 are covalently bound. As such, preferred chemotherapeutic agents contain a functional group such as an alcohol, acid, carbonyl, sulfhydryl (thiol) or amine group to be used in the conjugation to p97. Adriamycin is in the amine class and there is also the possibility to link through the carboxyl group as well. Paclitaxel (taxol) is in the alcohol class. Chemotherapeutic agents without suitable conjugation groups may be further modified to add such a group. All these compounds are contemplated in this invention. In the case of multiple therapeutic agents, a combination of various conjugations can be used.

“Increasing relative delivery” as used herein refers to the effect whereby accumulation at a site (such as an organ or a neoplasia) of a composition of the invention (i.e. a composition comprising a chemotherapeutic agent conjugated to p97) is increased relative to accumulation of a composition comprising the non-conjugated chemotherapeutic agent administered at an equivalent dose. This may be caused by increased specific or non-specific binding of the modified composition at the tumour site compared to the composition without a conjugated agent.

“Therapeutic index” means the dose range (amount and/or timing) above the minimum therapeutic amount and below an unacceptably toxic amount.

“Equivalent dose” means a dose that contains the same amount of active agent.

“Unacceptable cardiotoxicity” means a level of cardiotoxicity that is deemed unacceptable by a skilled analyst, and may vary depending on the patient.

“Brain tumours and other neoplasia in or around the brain or cancer of the brain” as used herein includes both primary tumours and/or metastases that develop in or around the brain. It may also mean metastases of brain tumours that migrate elsewhere in the body, but remain responsive to p97-chemotherapeutic agents. Many types of such tumours and neoplasia are known. Primary brain tumours include glioma, meningioma, neurinoma, pituitary adenoma, medulloblastoma, craniopharyngioma, hemangioma, epidermoid, sarcoma and others. 50% of all intracranial tumours are intracranial metastasis. As used herein, tumours and neoplasia may be associated with the brain and neural tissue, or they may be associated with the meninges, skull, vasculature or any other tissue of the head or neck. Such tumours are generally solid tumours, or they are diffuse tumours with accumulations localized to the head. Tumours or neoplasia for treatment according to the invention may be malignant or benign, and may have been treated previously with chemotherapy radiation and/or other treatments.

The term an “effective amount” or a “sufficient amount” of an composition as used herein means an amount sufficient to effect beneficial or desired results, including clinical results. For example, in the context of administering the composition to treat cancer an effective amount of the composition is, for example, an amount sufficient to achieve such a reduction in cancer cell proliferation or growth, a reduction in the progression of the cancer and/or an increased survival of the recipient as compared to the response obtained without administration of the composition.

As used herein, and as well understood in the art, “treatment or to treat” is an approach for obtaining beneficial or desired results, including clinical results. Beneficial or desired clinical results can include, but are not limited to, alleviation or amelioration of one or more symptoms or conditions of the cancer, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “animal” as used herein includes all members of the animal kingdom, preferably a mammal, more preferably human. When treating cancer the animal will have, be suspected of having or be predisposed to having a cancer of the brain.

### II. COMPOSITIONS AND PREPARATION THEREOF

The present invention generally provides methods and compositions comprising p97 linked to a chemotherapeutic agent for use in treating brain tumours and other neoplasia in and around the brain. The present invention also provides novel p97-chemotherapeutic agent conjugates along with modified forms of p97 and chemotherapeutic agents useful for preparing the conjugates of the invention.

In general, p97-chemotherapeutic agents can be prepared using techniques known in the art. There are numerous approaches for the conjugation or chemical crosslinking of compounds to p97 and one skilled in the art can determine which method is appropriate for the compound to be conjugated. The method employed must be capable of joining the chemotherapeutic agent with p97 without interfering with the ability of p97 to bind to its receptor, preferably without influencing the biodistribution of the p97-chemotherapeutic agent compared to p97 alone, and/or without significantly altering the desired activity of the compound once delivered. Methods of conjugating p97 to a various compounds include, for example, reacting an activated ester on a linker group attached to the chemotherapeutic agent directly with a free amino group on the p97 molecule (1-step reaction—Scheme 1). Alternatively, a reactive group, for example a maleimide, may react with free thiols that have been created on the p97 molecule via reaction with N-succinimidyl S-acetylthioacetamide (SATA) or through other groups where persons skilled in the art can attach them to p97 (²-step reaction—Scheme 1). Compounds may also be linked via a free carboxyl group on the p97 molecule by first activating the carboxyl group and then reaction with a free hydroxyl, amino or thiol group on a linker attached to the compound. A chemotherapeutic agent having for example, a free carboxyl group or a reactive amino, hydroxyl or thiol group, may also be conjugated directly to p97 using the 1-step or 2-step reactions described above.

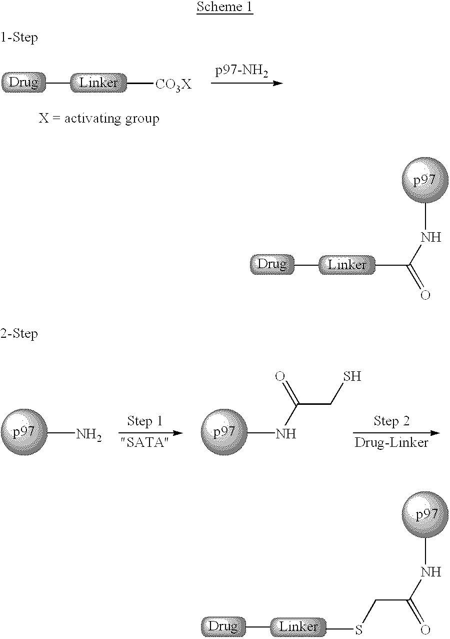

The linker is preferably an organic moiety constructed to contain an alkyl, aryl and/or amino acid backbone and which will contain an amide, ether, ester hydrazone, sulphide, disulphide linkage or any combination thereof. Linkages containing amino acid, ether and amide bound components will be stable under conditions of physiological pH, normally 7.4 in serum and 4-5 on uptake into cells (endosomes). Preferred linkages are linkages containing esters or hydrazones that are stable at serum pH but hydrolyse to release the drug when exposed to intracellular pH. Disulphide linkages are sensitive to reductive cleavage and amino acid linkers can be designed to be sensitive to cleavage by specific enzymes in the desired target organ. Particularly preferred linkages include an amide linkage between p97 and the linker group or between p97 and the chemotherapeutic agent. Exemplary linkers are set out in Blattler et al. Biochem, 24:1517-1524, 1985; King et al., Biochem. 25:5774-5779, 1986; Srinivasachar and Nevill, Biochem. 26:2501-2509, 1939. Preferred methods of conjugating p97 to a various compounds are set out in the example section, below. Particularly preferred for linking complex molecules to p97 is the SATA/sulfo-SMCC (sulfosuccinimidyl-4-N-maleimidomethyl-cyclohexane-1-carboxylate) cross-linking reaction (Pierce (Rockford, Ill.)).

New conjugates of p97 and chemotherapeutic agents have been prepared which incorporate the above-listed preferred linkages. The present invention therefore provides p97-chemotherapeutic agent conjugates selected from the group consisting of:

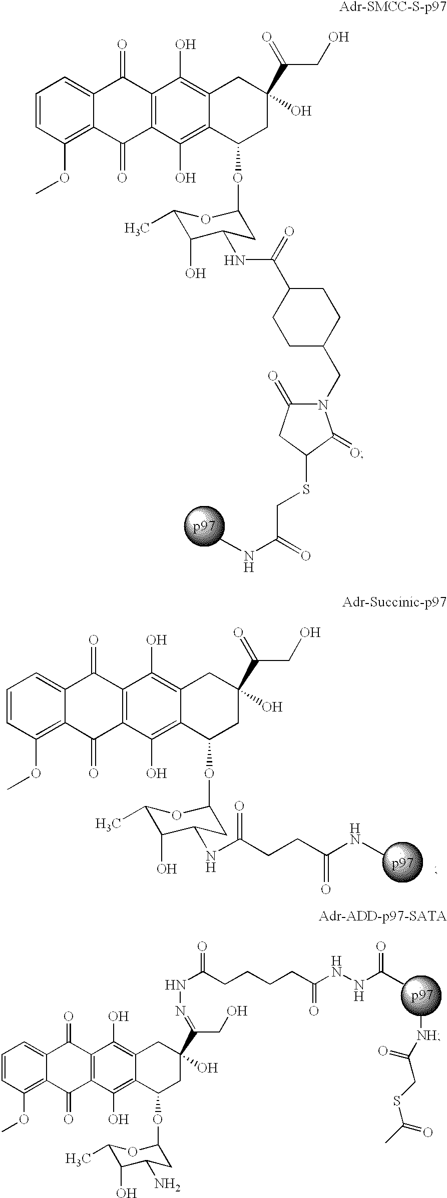

As previously mentioned, a preferred method of crosslinking p97 to chemotherapeutic agents involves the SATA/sulfo-SMCC crosslinking reaction. In an embodiment of the present invention, p97 is modified to incorporate one or more sulfhydryl (thiol) groups on its structure for participation in the SATA/sulfo-SMCC reaction. This has been accomplished by reacting p97 with N-succinimidyl S-acetylthioacetate (SATA) followed by deacelylation of the sulfhydryl group using, for example, hydroxylamine hydrochloride. The present invention therefore provides a modified p97 molecule in which one or more free amino (NH₂) groups have been converted to —NHC(O)CH₂SH groups (herein referred to as p97—SH).

Adriamycin has also been modified to incorporate within its structure a SMCC group for participation in the SATA/sulfo-SMCC crosslinking reaction. Modification of adriamycin in this manner may be accomplished by reacting adriamycin hydrochloride salt with SMCC in the presence of a base, preferably Hunig's base (diisopropylethylamine) in an inert solvent, for example dimethylformamide (DMF). The present invention therefore provides adriamycin-SMCC having the following structure:

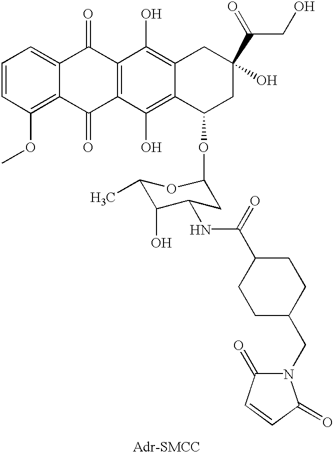

Other forms of adriamycin and other chemotherapeutic agents (including taxol) modified for linking to p97 that are included within the present invention may be found in the Examples hereinbelow.

In a further embodiment of the present invention theme is provided a method of preparing a p97-adriamycin conjugate comprising the steps of:

dissolving adriamycin in an inert solvent, preferably DMF, and adding an organic base, preferably triethylamine;

adding a solution of SMCC in an inert solvent, preferably DMF;

adding mercapto acetic acid;

adding a coupling reagent, preferably O-benzotriazol-1-yl-N,N,N′,N′-tetramethyluronium tetrafluoroborate (TBTU);

adding the solution of adriamycin, base, SMCC, mercaptoacetic acid and coupling reagent slowly to a solution of p97 and reacting under conditions to provide adriamycin-p97 conjugates; and

purifying the adriamycin-p97 conjugates.

For linking metals to p97, preferred reactions include, but are not limited to, binding to tyrosine residues through Chloramine T methods, or use of Iodo beads (Pierce) for iodination reactions. Such methods are well known in the art, but have not previously been employed with p97. p⁹⁷ may also be labeled with radioisotopes of, for example, technetium and rhenium. This may be accomplished, for example, by linking the succinimidyl hydrazine nicotinic hydrochloric (HYNIC) ligand (Abrams, et al. J. Nucl. Med 31:2022-2028, 1990), which is known to chelate radioisotopes of technetium and rhenium, to p97.

Methods for conjugating p97 with the representative labels set forth above may be readily accomplished by one of ordinary skill in the art (see, Trichothecene Antibody Conjugate, U.S. Pat. No. 4,744,981; Antibody Conjugate, U.S. Pat. No. 5,106,951; Fluorogenic Materials and Labeling Techniques, U.S. Pat. No. 4,018,884; Metal Radionuclide Labeled Proteins for Diagnosis and Therapy, U.S. Pat. No. 1,897,255; and Metal Radionuclide Chelating Compounds for Improved Chelation Kinetics, U.S. Pat. No. 4,988,496, see also Inman, Methods In Enzymology, Vol. 34, Affinity Techniques, Enzyme Purification: Part B. Jakoby and Wichek (eds.), Academic Press, New York, p. 30, 1974; see also Wilohek ad Bayer, “The Avidin-Biotin Complex in Bioanalytic Applications”, Anal. Biochem. 171:1-32, 1988; all incorporated herein by reference in their entirety for all purposes).

The therapeutic agent may also be linked to an antibody that binds to p97 for delivery to target sites. The preparation of antibodies to p97 is described hereinbelow.

If the chemotherapeutic agent is a protein or a peptide, there are many crosslinkers available in order to conjugate the compound with the p97 or a substance that binds p97. (See for example, Chemistry of Protein Conjugation and Crosslinking 1991, Shans Wong, CRC Press, Ann Arbor). The crosslinker is generally chosen based on the reactive functional groups available or inserted on the therapeutic compound. In addition, if there are no reactive groups a photoactivabible crosslinker can be used. In certain instances, it may be desirable to include a spacer between the p97 and the compound. In one example, p97 and protein therapeutic compounds can be conjugated by the introduction of a sulfhydryl group on the p97 and the introduction of a cross-linker containing a reactive thiol group on to the protein compound through carboxyl groups (see, Wawizynczak, E. J. and Thorpe, P. E. in Immunoconjugates: Antibody Conjugates in Radioimaging and Therapy of Cancer, C. W. Vogel (Ed.) Oxford University Press, 1987, pp. 28-55; and Blair, A. H. and T. I. Ghose, J. Immunol. Methods 59:129, 1983).

p97-chemotherapeutic agents can comprise one or more compound moieties linked to p97. For example, conjuagation reactions may conjugate from 1 to 10 or more molecules of adriamycin to a single p97 molecule, Particularly preferred ratios of p97 to adriamycin are 1:7 to 1:8. Several atoms of gold or iodine can be conjugated to a single p97 polypeptide. These formulations can be employed as mixtures, or they may be purified into specific p97:compound (mol:mol) formulations. Those skilled in the art are able to determine which format and which mol:mol ratio is preferred. Furdier, mixtures of compounds may be linked to p97, such as the p97-adriamycin-cisplatinum composition set out in the examples. These p97-chemotherapeutic agents may consist of a range of mol:mol ratios. These, too, may be separated into purified mixtures or they may be employed in aggregate.

A. Preparation of p97

The p97 peptide for use in the methods and compositions of the present invention may be obtained, isolated or prepared from a variety of sources.

In one aspect, standard recombinant DNA techniques may he used to prepare p97 or derivatives thereof. Within one embodiment, DNA encoding p97 may be obtained by polymerase chain reaction (PCR) amplification of the p97 sequence (see, generally, U.S. Pat. Nos. 4,683,202; 4,683,195; and 4,800,159; see, also, PCR Technology: Principles and Applications for DNA Amplification, Erlich (ed.), Stockton Press (1989)). Briefly, double-stranded DNA from cells which express p97 (e.g., SK-MEL-28 cells) is denatured by heating in the presence of heat stable Taq polymerase, sequence specific DNA primers such as 5′ GCGGACTTCCTCGG 3′ (SEQ ID NO: 1) and 5′ TCGCGAGCTTCCT 3′ (SEQ ID NO: 2)), ATP, CTP, GTP and TTP. Double-stranded DNA is produced when the synthesis is complete. This cycle may be repeated many times, resulting in a factorial amplification of p97 DNA. The amplified p97 DNA may then be readily inserted into an expression vector as described below.

Alternatively, DNA encoding p97 may be isolated using the cloning techniques described by Brown et al. in the UK Patent Application No. GB 2188637. Clones which contain sequences encoding p97 cDNA have been deposited with the American Type Culture Collection (ATCC) under deposit numbers CRL 8985 (PMTp97b) and CRL 9304 (pSVp97a).

Within one embodiment of the present invention, truncated derivatives of p97 are provided. For example, site-directed mutagenesis may be performed with the oligonucleotide WJ31 5′CTCAGAGGGCCGCTGCGCCC-3′(SEQ ID NO: 3) in order to delete the C-terminal hydrophobic domain beyond nucleotide 2219, or with the oligonucleotide WJ32 5′ CCA GCG CAG CTAGCGGGGGCAG 3′(SEQ ID NO: 4) in order to introduce an Nhe I site and a STOP codon in the region of nucleotides 1146-1166, and thereby also constructing a truncated form of p97 comprising only the N-terminal domain. Similarly, mutagenesis may also be performed on p97 such that only the C-terminal domain is expressed. Within one embodiment, Xho sites are inserted by mutagenesis with the oligolnucleotide WJ 5′-ACACCAGCGCAGCTCOAGGGOCAGCCG 3′(SEQ ID NO:5) into both the N-terminal and C-terminal domains, allowing subsequent deletion of the N-terminal domain. Various other restriction enzymes, including for example, Eco RI, may also be utilized in the context of the present invention in order to construct deletion or truncation derivatives of p97.

Mutations may be introduced at particular loci by synthesizing oligonucleotides containing a mutant sequence, flanked by restriction sites enabling the ligation of the mutated fragments to fragments of the native sequence. Following ligation, the resulting reconstructed sequence encodes a derivative having the desired amino acid insertion, substitution, or deletion. Alternatively, as noted above oligonucleotide-directed site-specific mutagenesis procedures may be employed to obtain an altered gene having particular codons altered according to the desired substitution, deletion, or insertion. Exemplary methods of making the alterations set forth above are disclosed by Sambrook et al. Molecular Cloning A Laboratory Manual, 2d Ed., Cold Spring Harbor Laboratory Press (1989).

Within a particularly preferred embodiment of the invention, p97 is cloned into an expression vector as a truncated cDNA with a deletion of the GPI anchor sequence located in the carboxy terminus of the protein.

Briefly, the p97 gene is generated by polymerase chain reaction (PCR) using the cloned p97 cDNA as a template. The truncated p97 is synthesized using WJ47, the 5′ PCR primer encompassing coordinates 36 to 60 (coordinates based on the cDNA map) and additionally containing a Sna BI restriction site. The sequence of WJ47 is 5′-GCG CTA CGT ACT CGA GGC CCC AGC CAG CCC CGA CGC CGC C-3′ (Seq ID: 6). The 3′ primer, WJ48, encompasses coordinates 2172 to 2193 and additionally contains both a TGA termination codon and a SaBI restriction site. The DNA sequence of WJ48 is 5′-CGC GTA CGT ATG ATC ATC AGC CCG AGC ACT GCT GAG ACG AC-3′ (Seq ID: 7. Following amplification, the truncated p97 product is inserted into pNUT_H (obtained from Palmiter (1986) PNAS 83:1261-1265 at the Sma I restriction site. The orientations of the resulting plasmids may be determined by PCR using one priming oligonucleotide that anneals to the vector sequence and a second priming oligonucleotide that anneals to the insert sequence. Alternatively, appropriate restriction digests can be performed to verify the orientation. Expression of the amplified sequence results in the production of a soluble p97 protein lacking the hydrophobic domain.

As noted above, the present invention provides recombinant expression vectors which include either synthetic, or cDNA-derived DNA fragments encoding p97 or derivatives thereof, which are operably linked to suitable transcriptional or translational regulatory elements. Suitable regulatory elements may be derived from a variety of sources, including, but not limited to, bacterial, fungal, viral, mammalian, and insect genes. Selection of appropriate regulatory elements is dependent on the host cell chosen, and may be readily accomplished by one of ordinary skill in the art. Examples of regulatory elements include, in particular, a transcriptional promoter and enhancer or RNA polymerase binding sequence, a ribosomal binding sequence, including a translation initiation signal. Additionally, depending on the host cell chosen and the vector employed, other genetic elements, such as an origin of replication, additional DNA restriction sites, enhancers, sequences conferring inducibility of transcription, and selectable markers, may be incorporated into the expression vector.

DNA sequences encoding p97 may be expressed by a wide variety of prokaryotic and eukaryotic host cells, including, but not limited to, bacterial, mammalian, yeast, fungi, viral, plant, and insect cells. Methods for transforming or transfecting such cells for expressing foreign DNA are well known in the art (see, e.g., Itakura et al., U.S. Pat. No. 4,704,362; Hinnen et al. (1978) PNAS USA 75:1929-1933; Murray et at., U.S. Pat. No. 4,801,542; Upshall et al., U.S. Pat. No. 4,935,349; Hagen et a., U.S. Pat. No. 4,78,950; Axel et at., U.S. Pat. No. 4,399,216; Goeddel et al., U.S. Pat. No. 4,766,075; and Sambrook et al., supra).

Promoters, terminators, and methods for introducing expression vectors of an appropriate type into, for example, plant, avian, and insect cells may be readily accomplished by those of skill in the art. Within a particularly preferred embodiment of the invention, p97 is expressed from baculoviruses (see, e.g., Luckow and Summers (1988) BioTechnology 6:47; Atkinson et al. (1990) Petic. Sci. 28:215-224). The use of baculoviruses such as AcMNPV is particularly preferred since host insect cells express the GPI-cleaved forms of p97. p97 may be prepared from cultures of the host/vector systems described above that express the recombinant p97. Recombinantly produced p97 may be further purified as described in more detail below.

The soluble form of p97 may be prepared by culturing cells containing the soluble p97 through the log phase of the cell's growth and collecting the supernatant. Preferably, the supernatant is collected prior to the time at which the cells lose viability. Soluble p97 may then be purified as described below, in order to yield isolated soluble p97. Suitable methods for purifying the soluble p97 can be selected based on the hydrophilic property of the soluble p97. For example, the soluble p97 may be readily obtained by Triton X-114 Phase Separation.

In another example, p97 may be isolated from cultured CHO cells genetically engineered to express the GPI-anchored p97. The GPI-anchored protein may be harvested by a brief incubation with an enzyme capable of cleaving the GPI anchor. Such enzymes are known in the art (Ferguson (1988) Ann. Rev. Bichem. 57:285-320) and representative examples are described supra. The cleaved soluble protein may be recovered from the medium, and the cells may then be returned to growth medium for further expression of the protein. Cycles of growth and harvest may be repeated until sufficient quantities of the protein are obtained. A particularly preferred GPI enzyme is phospholipase C (PI-PLC) which may be obtained either from bacterial sources (see, Low “Phospholipase Purification and Quantification” The Practical Approach Series: Cumulative Methods Index, Rickwood and Hames, eds. IRC Press, Oxford, N.Y. (1991); Kupe et al. (1989) Eur. J. Biochem. 185:151-155; Volwerk et al. (1989) J. Cell. Biochem. 39:315-325) or from recombinant sources (Koke et al. (1991) Protein Expression and Purification 2:51-58; and Henner et al. (1986) Nuc. Acids Res. 16:10383),

p9⁷ and derivatives thereof, including the soluble p97, may be readily purified according to the methods described herein. Briefly, p⁹7 may be purified either from supernatants contains solubilized p97, or from cultured host/vector systems as described above. A variety of purification steps, used either alone or in combination may be utilized to purify p97. For example, supernatants obtained by solubilizing p97, or from host/vector cultures as described above, may be readily concentrated using commercially available protein concentration filters, such as an Amicon or Millipore Pellicon ultrafiltration unit, or by “salting out” the protein followed by dialysis. In addition the supernatants or concentrates may be applied to an affinity purification matrix such as an anti-p97 antibody bound to a suitable support. Alternatively, an anion exchange resin, such as a matrix or substrate having pendant diethylaminoethyl (DEAE) groups, may be employed. Representative matrices include acrylamide, agarose, dextran, cellulose or other types commonly employed in protein purification. Similarly, cation exchangers which utilize various insoluble matrices such as sulfopropyl or carboxymethyl groups may be also used.

Finally, one or more reversed-phase high performance liquid chromatography (RP-HPLC) steps using hydrophobic RP-HPLC media, e.g. silica gel having pendant methyl or other alipathic groups, can be employed to further purify p97.

p97 fragments may also be generated using the techniques described above, with modifications well known in the art. For example, p97 expression vectors may be modified so that the expressed protein is a desired fragment of p97. This protein may be isolated from the expression system (i.e., extracted from cells), or it may be designed to be secreted into the supernatant of the expression system, and isolated using techniques described above. Alternatively, full length p97 protein may he generated and purified, and p97 fragments may then be generated by cleavage reactions designed to generate the desired fragment. Chemical synthesis is an alternative route to obtain the desired p97 protein or fragment thereof.

In the context of the present inventions “isolated” or “purified,” as used to define the purity of p⁹⁷, refer to a protein that is substantially free of other proteins of natural or endogenous origin and that contain less than about 5% and preferably less than about 1% by mass of protein contaminants due to the production processes. P97 may be considered “isolated” if it is detectable as a single protein band upon SDS-PAGH, followed by staining with Coomassie Blue.

B. Preparation of Antibodies to p97

Based on the teaching of the instant specification, antibodies to mouse or human p97 have many uses including, but not limited to, the use for the isolation and purification of p97, use in research and identification of p97 both in vitro and it vivo, and potential diagnostic and therapeutic uses. It is, therefore, useful to briefly set forth preferred antibodies to p97, and methods of producing such antibodies.

Antibodies reactive against p97 are well known in the art. Additional anti-p97 antibodies are provided by the present invention. Representative examples of anti-p97 antibodies include L235 (ATCC No. HB 8466; see, Real et al. (1985) Cancer Res. 45:4401 4411; see, also, Food et al. (1994) J. Biol Chem. 269(4): 3034-3040), 4.1, 8.2, 96.5 and 118.1 (see, Brown et al. (1981) J. Immunol. 127(2):539-546; and Brown et al. (1981) Proc. Natl. Acad. Sci. USA 78(1):539-543); and HybC (Kennard et al. (1996) Nat. Med. 2(11):1230-1235). Other monoclonal antibodies, including, but not limited to, 2C7 and 9B6, have been generated at Synapse Technologies Inc. Antibodies to the mouse p97 include, for example, a rabbit anti-human p97 polyclonal antibody generated against a fragment of the mouse p97. In the context of the present invention, antibodies are understood to include, for example, monoclonal antibodies, polyclonal antibodies, antibody fragments (e.g., Fab, and F(ab′)2) and recombinantly produced binding partners. Antibodies are understood to be reactive against p97 if the Ka is greater than or equal to 10⁻⁷ M.

Polyclonal antibodies may be readily generated by one of ordinary skill in the art from a variety of warm-blooded animals. Monoclonal antibodies may also be readily generated using conventional techniques (see, e.,g. U.S. Pat. Nos. RE 32,011, 4,902,614; 4,543,439; and 4,411,993; see, also, Kennett, McKearn, and Bechtol (eds.) Monoclonal Antibodies, Hybridomas: A New Dimension in Biological Analyses, Plenum Press, (1980); and Harlow and Lane (eds.) Antibodies: A Laboratory Manual, Cold Spring Harbor Laboratory Press (1988)). Preparation of preferred antibodies is further described in the example section, below.

### III. METHODS OF USING COMPOSITIONS

The present invention includes the use of a p97-chemotherapeutic agent composition of the invention to treat brain tumours and other neoplasia in and around the brain; to increase the survival of an animal having a tumour or neoplasia in and around the brain; to reduce the growth or proliferation of a brain tumour or neoplasia in and around the brain; to reduce the toxicity of a chemotherapeutic agent; to increase the delivery of a chemotherapeutic agent to the brain and to target a chemotherapeutic agent to the brain.

Accordingly, the present invention provides a method of treating a brain tumour or other neoplasia in and around the brain comprising administering an effective amount of a composition comprising a chemotherapeutic agent conjugated to p97 to an animal in need thereof. The invention also provides a use of a composition comprising a chemotherapeutic agent conjugated to p97 to prepare a medicament to treat a brain tumour or other neoplasia in and around the brain.

The cancer can be any brain tumour or other neoplasia in or around the brain including both primary brain cancers and metastases. Examples of brain cancers include, but are not limited to, glioma, meningioma, neurinoma, pituitary adenoma, medulloblastoma, craniopharyngioma, hemangioma, epidermoid, and sarcoma.

One of skill in the art can readily determine if a p97-chemotherapeutic agent composition may be useful in treating cancer using techniques known in the art. For example, the composition may first be tested in an in vitro system and subsequently tested in an animal model. The animal model may be as described in Examples 11 and 12.

The inventors have shown that there is an increased survival of mice with brain tumours when a chemotherapeutic agent is conjugated to p97 as compared to when the chemotherapeutic agent is administered alone, Accordingly, the present invention provides a method of increasing the survival of an animal having a brain tumour or other neoplasia localized in or around the brain comprising administering an effective amount of a composition comprising a chemotherapeutic agent conjugated to p97 to an animal in need thereof. The invention also includes a use of a composition comprising a chemotherapeutic agent conjugated to p97 to prepare a medicament to increase the survival of an animal with a brain tumour or other neoplasia in and around the brain.

The invention also includes a method for increasing delivery of a chemotherapeutic agent to a brain tumour or neoplasia localized in or around the brain, said method comprising administering a p97-chemotherapeutic agent to an animal having a brain tumour or neoplasia in or around the brain, wherein the amount of chemotherapeutic agent delivered as part of the p97-chemotherapeutic agent to said neoplasia is increased relative to delivery of the chemotherapeutic agent when said chemotherapeutic agent is not conjugated to p97 and administered at an equivalent dose. The invention also includes a use of a composition comprising a p97-chemotherapeutic agent to prepare a medicament to increase the delivery of a chemotherapeutic agent to a brain tumour or neoplasia localized in or around the brain.

The invention further includes a method for increasing delivery of a chemotherapeutic agent to a brain tumour or neoplasia localized in or around the brain, said method comprising:

a) conjugating a chemotherapeutic agent to p97 to generate a p97-chemotherapeutic agent; and

b) administering said p97-chemotherapeutic agent to an animal having a neoplasia in or around the brain, wherein the amount of chemotherapeutic agent delivered as part of the p97-chemotherapeutic agent to said neoplasia is increased relative to delivery of the chemotherapeutic agent when said chemotherapeutic agent is not conjugated to p97 and administered at an equivalent dose.

The invention yet also includes a method for targeting a chemotherapeutic agent to a neoplasia localized in or around the brain, said method comprising administering a p97-chemotherapeutic agent to an animal having a neoplasia localized in or around the brain, wherein said patient experiences increased delivery of said chemotherapeutic agent to said neoplasia compared to when the chemotherapeutic agent is not conjugated to p97 and is administered at an equivalent dose. The invention also includes a use of a composition comprising a p97-chemotherapeutic agent to prepare a medicament to target a chemotherapeutic agent to a neoplasia localized in or around the brain.

The invention further includes a method for targeting a chemotherapeutic agent to a neoplasia localized in or around the brain, said method comprising;

a) conjugating a chemotherapeutic agent to p97 to generate a p97-chemotherapeutic agent; and

b) administering the p97-chemotherapeutic agent to an animal having a neoplasia localized in or around the brain, wherein said patient experiences increased delivery of said chemotherapeutic agent to said neoplasia compared to when the chemotherapeutic agent is not conjugated to p97 and is administered at an equivalent does.

The inventors have also demonstrated that conjugating a chemotherapeutic agent to p97 reduces the systems toxicity of the chemotherapeutic agent. Accordingly, the present invention provides a method of reducing the toxicity of a chemotherapeutic agent comprising administering an effective amount of a composition comprising a chemotherapeutic agent conjugated to p97 to an animal in need thereof. The invention also includes a use of a composition comprising a chemotherapeutic agent conjugated to p97 to prepare a medicament to reduce the toxicity of the chemotherapeutic agent.

In this embodiment, preferred chemotherapeutic agents are those, which in the free form, demonstrate unacceptable systemic toxicity at desired doses. The general systemic toxicity of these agents is reduced by linkage to p97. Particularly preferred are cardiotoxic compounds that are useful therapeutics but are dose limited by cardiotoxicity. A classic example is adriamycin (also known as doxorubicin) and its analogs, such as daunorubicin. Linking p97 to such drugs effectively prevents accumulation and associated cardiotoxicity at the heart.

In addition to chemotherapeutic agents, the p97 may be conjugated to other agents such as radioimaging agents including radiolabeled technetium or rhenium such as Technetium-99-m (Te-99m). Such agents can be used for diagnostic imaging of a cancer in the brain. Accordingly, the present invention provides a method of detecting or diagnosing a brain tumour or other neoplasia localized in or around the brain comprising administering an effective amount of a composition comprising a radioimaging agent conjugated to p97 to an animal in need thereof.

Compositions of the present invention may be administered encapsulated in or attached to viral envelopes or vesicles, or incorporated into cells. Vesicles are micellular particles which are usually spherical and which are frequently lipidic. Liposomes are vesicles formed from a bilayer membrane. Suitable vesicles include, but are not limited to, unilamellar vesicles and multilamellar lipid vesicles or liposomes. Such vesicles and liposomes may be made from a wide range of lipid or phospholipid compounds, such as phosphatidylcholine, phosphatidic acid, phosphatidylserine, phosphatidylethanolamine, sphingomyelin, glycolipids, gangliosides, etc. using standard techniques, such as those described in, e.g., U.S. Pat. No. 4,394,448. Such vesicles or liposomes may be used to administer compounds intracellularly and to deliver compounds to the target organs. Controlled release of a p97-composition of interest may also he achieved using encapsulation (se, e.g., U.S. Pat. No. 5,186,941).

Any route of administration which dilutes the composition into the blood stream may be used. Preferably, the composition is administered peripherally, most preferably intravenously or by cardiac catheter. Intra-jugular and intra-carotid injections are also useful. Compositions may be administered locally or regionally, such as intra-peritoneally. In one aspect, compositions are administered with a suitable pharmaceutical diluent or carrier.

Dosages to be administered will depend on individual needs, on the desired effect, and on the chosen route of administration. Preferred dosages of p97 range from about 0.2 pmol/kg to about 2.5 nmol/kg, and particularly preferred dosages range from 2-250 pmol/kg; alternatively, preferred doses of p97 may be in the range of 0.02 to 2000 mg/kg. These dosages will be influenced by the number of compound moieties associated with each p97 molecule. Alternatively, dosages may be calculated based on the compound administered. Doses of p97-adriamycin comprising from 0.005 to 100 mg/kg of adriamycin are also useful in viva. Particularly preferred is a dosage of p97-adriamycin comprising from 0.05 mg/kg to 20 mg/kg of adriamycin. Those skilled in the art can determine suitable doses for other compounds linked to p97 based on the recommended dosage used for the free form of the compound. p97 generally reduces the amount of drug needed to obtain the same effect. Additionally, p97 increases the maximum tolerated doses of these compounds because of the protective effect it has on the biodistribution. Those skilled in the art know how to select suitable dosages based on these and other considerations.

The following non-limiting examples are illustrative of the present invention:

### EXAMPLES

### Example 1

### Generation of a p97 Expression System in BHK Cells

Soluble p97 was obtained from a BHK TKneg (baby hamster kidney, thymidine kinase negative) (ATCC CRL 1632) cell line transfected with human p97 cDNA having a stop codon introduced at amino acid position 711 (glycine). The introduction of this stop codon resulted in the deletion of the GPI anchor attachment sequence. The cDNA was cloned into the expression vector pNUTΔH containing the DHFR gene allowing for selection with methotrexate. Transfection was performed using lipofectin, and selection was carried out in 0.5 mM methotrexate. Clones were screened for p97 production by FACS analysis and immunoprecipitation.

### Example 2

### Preparation and Purification of Human 97

Purified recombinant secreted human p97 was produced from transfected BHK cells. A BHK culture supernatant containing secreted p97 was first prepared (see part A) and p97 was then purified from the obtained BHK culture supernatant (see part B). Techniques used herein are described in Kennard et al. (1993) Biotech. Bioeng. 42:480-86; and Food et al. (1994) J. Biol. Chem. 269:3034-40.

A. Production of BHK Cell Medium Containing Recombinant Secreted Human p97

Cell line: The BHK TKneg (baby hamster kidney, thymidine kinase negative) (ATCC CRL 1632) cell line transfected with human p97 as described in Example 1 and selected with 0.5 mM methotrexate was used. Clones were screened for p97 production by FACS analysis and immunoprecipitation.

Materials: The following materials were supplied by various commercial suppliers such as Gibco BRL, Faulding, etc. The BHK culture medium contained 1 M HEPES stock solution, 1 M Sodium azide stock solution, 100 mM Zinc sulphate stock solution, DMEM/Ham's F-12, Fetal Bovine Serum (FBS), N-[2-Hydroxyethyl]piperazine-N′[2-ethanesulphonic acid]) (HEPES), L-glutamine 100×, Zinc sulphate (ZnSO₄7H₂O), Methotrexate (25 mg/ml), Phosphate buffered saline (PBS), Tryptan blue, Sodium azide, 0.05% Trypsin solution in 0.25 mM EDTA, EDTA. The following solutions were prepared: 500 ml of BHK culture media (DMEM/Ham's F-12 with additives); 100 ml of 1 M HEPES; 500 ml of 1 M sodium azide; 500 ml of 100 mM zinc sulphate.

Methods: Adherent cells frozen at −135° C. at a density of 1×10⁷ cells/m were transfected. A 1 ml aliquot of frozen cells was rapidly thawed in warm water with shaking. 9 ml of culture medium were added to thawed cells in a 15 ml centrifuge tube drop by drop to reduce the effects of medium change. The cells were then allowed to stand for ten minutes at room temperature and then centrifuged at 1000 rpm (230×G) for 5 minutes at 4° C. The supernatant was carefully removed and the cell pellet was resuspended in 10 ml of culture medium and added to a 25 cm² T flask. The cells were counted and the viability determined. The trypan exclusion method was used and the cells were counted using a haemocytometer. Incubation at 37° C. in a 5% CO₂ humidified atmosphere was carried out until the cells became confluent. The supernatant was then removed and the cells washed by adding 25 ml of PBS. After pouring off the PBS, the cells were removed by adding 1 ml of a 0.05% trypsin solution in 0.25 mM EDTA and incubated at 37° C. for 2 minutes in the CO₂ humidified incubator. The trypsin was immediately neutralized by adding 5 ml of culture medium. The cells were recovered from the T-flask surface by gently tapping the sides of the T-flask and pipetting the supernatant with a 10 ml pipette. The supernatant with the resuspended cells was recovered and placed in a sterile 15 ml polypropylene centrifuge tube and centrifuged at 1000 rpm (230×G) for 5 minutes at 4° C. After discarding the supernatant, the cells were resuspended in 10 ml of fresh culture medium. The cells were counted using the trypan blue exclusion method and a haemocytomoter. The cell culture was then scaled up to a 175 cm² T-flask by adding 50 ml of fresh culture medium.

The number of cells to be added to the 50 ml of culture medium in order to obtain a seeding density of 1-2×10⁵ cells/ml was determined. [For example, if the 10 ml cell suspension had 2×10⁶ cells/ml then (50×2×10⁵)/2×10⁶=5 ml were added]. The cells were incubated until confluence at 37° C. in a 5% CO₂ humidified atmosphere. Again, cells were counted using the trypan blue exclusion method and a hemocytometer. For the final scale up, a 11 roller bottle was seeded with the cells from the 175 cm² T-flask. The 50 ml of supernatant were poured off and the cells were then removed by adding 10 ml of a 0.05% trypsin solution in 0.25 mM EDTA and incubating at 37° C. for 2 min in the CO₂humidified incubator. The trypsin was immediately neutralized by adding 50 ml of culture medium. The cells were recovered from the T-flask surface by gently tapping the sides of the T-flask and pipetting the supernatant with a 25 ml pipette. The supernatant with the resuspended cells was recovered and placed in a sterile 50 ml polypropylene centrifuge tube, centrifuged at 1000 rpm (230×G) for 5 minutes at 4° C. The supernatant was discarded and the cells resuspended in 25 ml of fresh culture medium. The cells were counted using the trypan blue exclusion method and a haemocytometer.

The cultured was scaled up to a 11 roller bottle by adding 300 ml of fresh culture medium. The number of cells to be added to the 300 ml in order to obtain a seeding density of 1-2×10⁵ cells/ml was determined. [For example, if the 25 ml cell suspension had 1×10 ⁷ cells/ml, then (300×2×10⁵)/1×10⁷=6 ml were added]. Incubation was carried out until confluence at 37° C. in a 5% CO₂ humidified atmosphere. Cells were counted using the trypan blue exclusion method and a hemocytometer. The roller bottles were aerated daily with 5% CO₂ balance air and incubated at 37° C. The p97 secretion was monitored every two days using the Pandex assay method. All data was recorded in the worksheets. After approximately 7 to 10 days of culture, when the cells reached confluence, an additional 300 ml of culture medium were added. After a further 5 to 7 days of culture, the 600 ml of supernatant were recovered.

Since the cells were still viable and attached to the roller bottle, the culture may be re-fed with 300 ml of fresh culture medium, and topped up with a further 300 ml of culture medium after 3-5 days. The second 600 ml of supernatant were recovered after a further 3-5 days of culture. Following this protocol, 1200 ml of supernatant with secreted p⁹⁷ were recovered.

For recovering the p97 supernatant, the supernatant was centrifuged at 300 rpm (2056×g) for 10 min at 4° C. and the resulting supernatant was collected. The p97 concentration in the supernatant was determine (e.g., using a Pandex assay protocol). When necessary, the supernatant was concentrated 5 fold using a 30,000 MW cut-off ultrafiltration membrane. Preferably, the p97 concentration was >100 μg/ml. 20 mM sodium azide were added to the concentrated supernatant which was stored at 4° C. until p97 purification.

For quality control determinations, once in the roller bottles, the BHK cultures were monitored every two days for p9⁷ concentration. Typically, the concentration reached˜100 μg/ml. When this concentration was not achieved, the cell line was checked for mycoplasma contamination and the culture restarted from the first step. Cultures were checked for bacterial and yeast contaminations. If any contamination was detected, the culture was abandoned and rested from the first step.

Procedures for the Recovery and Purification of the Secreted p97 from the p97 Transfected BHK Culture Supernatant

Reagents: 3 ml affinity columns were prepared with immobilized L235 on AffiGel 10 (see, Example 3, below); Elution buffer (0.1M citric acid, pH 2.5); Neutralization buffer (1M HEPES, pH 9.0); Column storage solution (PBS, 20 mM sodium azide). 1M Sodium azide stock solution; Citric acid (C₆H₈O₇); (N-[2-Hydroxyethyl]piperazine-N′[2-ethanesulphonic acid]) (HEPES); Sodium azide; Phosphate buffered saline (PBS). The following solutions were prepared: 500 ml of buffer of citric acid at 0.1 M 500 ml of 1M HEPES Neutralization buffer; 500 ml of storage solution (To 490 ml PBS add 10 ml of the stock 1 M sodium azide solution to give a 20 mM solution of azide in PBS); 500 ml of a 1 M stock azide solution.

Methods: The BHK culture supernatant containing secreted p97 prepared as described supra was purified. To purify approximately 100 ml of supernatant, a 3 ml of column of L235 immobilized on AffiGel 10 was used (see Example 3, below), The concentration of p97 in the solution to be purified was determined using a method such as a Pandex assay. The column storage solution was drained off under gravity, and the column was washed with 15 ml of PBS, by allowing the PBS to flow through the column under gravity. The sample was passed through the column at 15-18 ml/hr at room temperature and allowed to flow through under gravity. When necessary, the flow was adjusted using a drain valve attached to the column. The eluate was collected and saved for testing for p97 concentration determination using the Pandex assay method. (This was used to monitor the efficiency of the column). Following a wash with 15 ml or PBS (saved for p97 determination using the Pandex assay method), the buffer was allowed to flow through under gravity. Six 5 ml tubes were placed in a rack and labeled 1 to 6. p97 was eluted with 15 ml of Elution buffer and 3 ml fractions were collected in 5 ml tubes. The buffer was allowed to flow through the column under gravity and the fractions were neutralized with the neutralization buffer to pH 7.0±0.4. The pH was rapidly checked by testing 20 μl samples on pH strips in the range pH 5-10. Fractions were monitored by absorbance at 280 nm. The majority of p97 was eluted in fractions 2 and 3. These fractions were usually pooled and the p97 concentration determined using a method such as a Pandex assay method. The column was washed with 15 ml of PBS and stored in 10 ml column storage solution. Columns were stable for up to 1 year at 4° C.

For quality control tests, the purity of p97 was determined. The following standard assays were performed to characterize the p97 produced and to determine whether the produced p97 was at least 98% pure. Batches falling below the standard were discarded. Purity was determined by SDS-PAGE, Western blot, LC MS, or GC Mass Spectrometry, The concentration was determined by OD (extinction coefficient). Immunofluorescence assays (Pandex), and amino acid composition analysis were also performed. The identity was determined by Tryptic digest and MALDI-TOF MS and tho reactivity by immunofluorescence assays.

Determination of p⁹⁷ concentration (˜1 mg/ml) was carried out by OD, extinction coefficient εl%@280=12 cm⁻¹, by Pandex assay and by amino acid composition.

### Example 3

### Production of an Anti-197 Affinity Column

A method for preparation of an AffiGel column with L235 antibody for use in the purification of secreted recombinant p97 from BHK cell supernatant was designed. First, L235 anti-human p97 monoclonal antibodies were produced using the I.235 hybridoma cell line. The L235 antibodies were then used to prepare an Affi-gel separation column. An alternative anti-p97 antibody HybC was also produced.

A. Production of L235 Anti-human p97 Monoclonal Antibodies

Cell lines: The Hybridoma L235 -ATCC. HB8446 L235 (M-19) cell line was used for producing the L235 antibodies. For the feeder layer, irradiated mouse embryonic fibroblast cells -ATCC X-56 were used.

The following items were supplied by standard commercial suppliers such as Gibco, EM Science, Sigma, BDH, etc. REPMI; Hybridoma medium; 1 M Sodium azide stock solution; 1M HEPES sock solution; 50 mM β-mercaptoethanol stock solution; Fetal Bovine Serum (FBS): (N-[2-Hydroxyethyl]piperazine-N′[2-ethanesulphinic acid]) (HEPES); non-essential amino acids 100×; L-glutamine and Pen/Strep 100×; L-proline 100×; β-mercaptoethanol; Phosphate buffered saline (PBS 10×); Trypan blue; Sodium azide. The following solutions were prepared 100 ml of 1 M HEPES; 500 ml of 1 M sodium azide; 100 ml of 50 mM β-mercaptoethanol.

500 ml of hybridoma and feeder layer culture media were prepared and the pH was adjusted to 7.4±0.2. 500 ml of RPMI solution were prepared from the powder according to the manufacturer's instructions. The powder was emptied into 1 l beaker with a stirrer bar and 500 ml of DDH₂O were added and mixed at room temperature. If necessary, the pH was adjusted, using either 1 M hydrochloric acid or 1 M sodium hydroxide. In a 1 l glass beaker with a stirrer bar, at room temperature, 425 ml of freshly prepared RPMI were added as well as:

50 ml of FBS (heat inactivated at 57° C. for 1 hr in a water bath)

10 ml of 1 M HEPES

5 ml nonessential amino acids 100×

5 ml L-glutamine 100×

5 ml L-proline 100×

0.5 μl 50 mM β-mercaptoethanol

After mixing at room temperature for −10 min, the medium was sterile filtered through a 0.22 μm filter under vacuum in a laminar flow hood and stored in a sterile 500 ml media bottle at 4° C. for up to 1 month.

The feeder cells were obtained from ATCC in polystyrene tubes with screw tops. The following steps were carried out in a laminar flow hood. A 1 ml aliquot of frozen cells was thawed rapidly in warm water with shaking. The thawed feeder layer cells were added to 50 ml of medium in a 50 ml polypropylene centrifuge tube and allowed to stand for ten minutes at room temperature. 2 ml of the cell suspension were added to 2×25 cm² T-flasks. 23 ml of the cell suspension were added to 2×150 cm² T-flasks. The cells were cultured for 1 day at 37° C. in a 50 CO₂ humidified atmosphere. The medium was poured off into a glass breaker and replaced with fresh culture medium—10 ml in the 25 cm² T-flask, 50 ml in the 150 ml T-flask. The cells were then cultured for another day at 37° C. in a 5% CO₂ humidified atmosphere.

For the hybridoma culture, a 1 ml aliquot of frozen cells was thawed rapidly in warm water with shaking. 9 ml of medium were added to the thawed cells in a 15 ml centrifuge tube drop by drop to reduce the effects of medium change and the cells were allowed to stand for ten minutes at room temperature.

Following a certification at 1000 rpm (230×g) for 5 minutes at 4° C., the supernatant was carefully discarded and the cell pellet resuspended in 10 ml of conditioned medium from the 175 cm² T-flask and added to a 25 cm² T-flask containing only the feeder layer. The cells were counted under the microscope using a haemocytometer and the viability determined using the trypan blue dye exclusion method. Following an incubation at 37° C. in a 5% CO₂ humidified atmosphere until the cell density reaches 1×10⁶ cells/ml and the viability >90%, viability was determined again using the trypan blue dye exclusion method and the cells were counted under the microscope using a haemocytometer. 10 ml of cells were transferred to the 175 cm² T-Flask containing the feeder layer and 100 ml of culture medium. The 25 cm² T-flask culture was kept in order to reseed another 175 cm² T-flask culture. The approximate viable cell density of the cells was determined to be about 2×10⁵ cells/ml, using the trypan blue dye exclusion method and counting the cells under the microscope using a haemocytometer. The cells in the 175 cm² T-flask culture were monitored until the viability of the hybridomas fell below 60-70%. Again, the trypan blue dye exclusion method was used and the cells were counted under the microscope using a haemocytometer. The cell density and viability was ideally determined every 2 days. The antibody concentration was also measured every 2 days using the monoclonal assay. The supernatant containing cells was removed and centrifuged at 1000 rpm (230×g) for 10 min at 4° C. and the cell free supernatant recovered (approximately 1×10⁶ cells/ml were left in the T-flask for the next culture—the feeder layer may be used for approximately 4 cell cultures) Add 20 mM sodium azide to the supernatant and store at 4° C. prior to antibody purification.

For quality control tests, the culture were monitored every 2 days for cell viability and density, as well as the concentration of secreted monoclonal antibody. When the antibody concentration was not ˜10 μg/ml when the cell density reached approximately 1×10⁶ cells/ml, the culture was abandoned and restarted. The cultures were checked for bacteria and yeast contaminations. If any contamination was detected, the culture was abandoned and restarted.

Further details on all procedures are described in the Antibody Handbook. Purity measurement were preferably performed using SDS-PAGE, IEF gel or LC. The concentration was typically determined using OD measurements or immunofluorescence assays (Pandex), and the affinity was evaluating by detecting p97 in Western blots or by using an ELSIA titration method.

B. Preparation of an Affinity Column Using L235 Antibody (L235 Immobilized AffiGel 10 Column or L235 Affinity Column)

The purified L235 was provided in a buffer containing 0. 1 M glycine HCl and 0.1 M Tris-HCl. The L235 was first transferred into a buffer containing 100 mM HEPES at pH 7.4±0.2 in a 15 ml Slide-A-Lyze cassette with 3 changes of HEPES with ˜24 hr between changes. L235 was concentrated to 15 mg/ml using 3 ml Centriprep or 15 ml Centricon concentrators (30,000 MW cut-off according to the manufacturer's instructions.

To prepare a 3 ml L235 affinity column, 6 ml of AffiGel- 10 (BioRad) suspension (˜50/50 solution) were transferred to an empty 1 cm diameter glass column and drained, The column was washed with 15 ml of cold (4° C.) dd H₂O, which were allowed to flow through the column under gravity. The bottom of the column was sealed with Parafilm and 3 ml of 15 mg/ml of L235 in 100 mM HEPES were added. The top of the column was sealed with Parafilm and the column was placed on a rocker at 4° C. for 4 hours with gentle rocking so the antibody mixed well with the gel. The column was drained and the solution saved to check for efficiency of antibody binding. The concentration of any unbound antibody in the elute was determined by the Pandex antibody assay method. The column was washed with 30 ml of PBS which were allowed to flow through the column under gravity. The column was stored at 4° C. in 15 ml of column storage solution.

For quality control, the antibody binding efficiency was determined as follows. The OD of the L235 solution was measured at 280 nm before and after contact with the AffiGel. The % efficiency was determined using the following equation:

(Dilution×OD of 15 mg/ml of L235×sample volume)−(Dilution×OD eluate×sample volume)×100

(Dilution×OD of 15 mg/ml of L235×sample volume)

The efficiency was ideally >75%

C. Alternative Method for Purification of p97 Using a HybC Antibody Affinity Column

A HybC anti-human p97 monoclonal antibody was produced by culturing the HybC hybridoma cell line. This antibody was used as an alternative for L235 for the purification of p97 from BHK cell supernatants.

Cell line: Hybridoma C -33B6E4 produced by Dr. Shuen-Kuei Liao (Dept. Pathology and Pediatrics, McMaster University, Hamilton Ont.)

For the hybridoma medium, 500 ml of DMEM solution were prepared from powder according to the manufacturer's instructions. The powder was emptied into a 1 l beaker with a stirrer bar and, after adding 500 ml of ddH₂O the solution was mixed at room temperature. The pH was checked to be ˜7.4±0.2 and adjusted if necessary, using either 1M hydrochloric acid or 1M sodium hydroxide. In a 1 l beaker with a stirrer bar at room temperature, 430 ml of freshly prepared DMEM were added, as well as 50 ml FBS (heat inactivated at 57° C. for 1 hr in a water bath), 10 ml of 1M HEPES, 5 ml L-Glutamine Pen/Strep, 5 ml non essential amino acids and 0.5 ml of 50 mM β-mercaptoethanol. The medium was mixed at room temperature for ˜10 min, sterile filtered through a 0.22 μm filter under vacuum in a laminar flow hood and stored in a sterile 500 ml media bottle at 4° C. for up to 1 month.

A 1 ml aliquot of frozen cells was rapidly thawed in warm water with shaking. 9 ml of medium were added to the thawed cells in a 15 ml centrifuge tube drop by drop to reduce the effects of medium change. The cells were allowed to stand for ten minutes at room temperature and centrifuged at 1000 rpm. (230×g) for 5 minutes at 4° C. The supernatant was carefully removed and the cell pellet was resuspended in 10 ml of culture medium and added to a 25 cm² T-flask to count the cells and determine the viability using the trypan blue exclusion method and counting the cells using a haemocytometer. Following incubation at 37° C. in a 5% CO₂ humidified atmosphere until the viable cell density reached 1×10⁶ cells/ml, the cell density and viability were determined as described above. The volume was scaled up to 50 ml by transferring the 10 ml contents of the 25 cm² T-flask to 75 cm² T-flasks and adding 40 ml of culture medium. The viable cell density was allowed to reach 1×10⁶ cells/ml. For the final scale up, 50 ml at 1×10⁶ cells/ml contents of the 75 cm² T-flask were used to inoculate 500 ml of media in a sterile 1 l spinner flask (inoculation viable cell density at −1-2×10⁵), The inoculation cell density was checked as described supra. The cells were cultured for approximately 10 to 15 days at 37° C. in a 5% CO₂ humidified atmosphere until the cell viability fell below 80%. The cells density and viability was measured every 2 days, as described supra. The antibody concentration was also measured every 2 days using the monoclonal antibody assay and the data was recorded in the worksheets. The supernatant containing cells was removed and centrifuged at 1000 rpm (230×g) for 10 min at 4° C. to pellet the cells. The cell free supernatant was carefully recovered and 20 mM sodium azide were added. The supernatant was stored at 4° C. prior to antibody purification. Quality control was tested as described supra.

### Example 4

### Preparation of Apo and Holo P97

FeCl₃ or ⁵⁵FeCl₃ may be used depending on the objectives of the study.

p97 (also called melanotransferrin; MTf) was concentrated by spinning 2 ml of purified MTf solution in a Centricon 30 tube for 12 min at 2000×g. The filtrate was saved and the filter was washed with 100 μl of filtrate for 5 min at 500×g. The concentration of the retentate was measured at 280 nm using the filtrate as blank. The molarity (moles/l) and concentration (mg/ml) were calculated with molar extinction coefficient (94420 abs 1 mole⁻¹) and (1.218 abs ml mg⁻¹). The concentrations and volume of the retentate were recorded.

A. Apo MTf

Fe and other metals were removed from MTf by dialysis as follows: 2 l of 0.1 M sodium acetate buffer pH 5.0, 0.001 M sodium citrate, 0.001 M EDTA were prepared. A Slide-A-Lyzer 10,000 MWCO dialysis cassette were hydrated in buffer for 30 s and the MTf retentate was introduced into cassette and dialyze in buffer for 3 h. Change buffer to 2 l 0.1 M NaCl, 0.040 NaHCO₃ and dialyze for 1-2 h. Recover dialysate and record volume. Measure concentration at 280 mm with dialysis buffer as blank. Record concentration. Concentrate if necessary.

B. Holo MTf

Prepare 5 mM FeCl₃ or ⁵⁵FeCl₃ in 0.5 M HCl, 25 mM sodium citrate, and 1 M NaHCO₃. Chelate iron with citrate: Add 25 μl FeCl₃ or ⁵⁵FeCl to 50 μl sodium citrate vortex and wait 15 min. Add 20 μl NaHCO₃ wait 15 min vortex periodically. Exhaust CO₂ released from solution. Add 250 μl of MTf (2 mg/ml) vortex and wait 1 h. Add 1 ml 100 mM NaCl, 20 mM NaHCO₃ and introduce into hydrated Slide-A-Lyzer 10,000 MWCO. Dialyze against 2 l of 100 mM NaCl, 20 mM NaHCO₃ for 1-2 h. Recover solution from dialysis cassette record volume, and measure concentration at 280 nm with using dialysis buffer as blank. Concentrate if required.

### Example 5

### Linking p97 to Chemotherapeutic Agents

These examples set forth methods of linking p97 to chemotherapeutic agents. A wide variety of p97-chemotherapeutic agents have been prepared.

### Example 5a

### Preparation of Starting Materials:

A. p97-SATA

A 250-mL rounded-bottomed flask equipped with a magnetic stirrer was charged with p97 (lot#13A, O.D.=1.60, C=1.314 mg.mL, 100 mL, 1.39e-3 mmol). The solution was stirred and a mixture of N-succinimidyl S-acetylthioacetate (SATA from Pierce, 96 mg, 0.417 mmol, 300 equiv.) in dimethyl sulfoxide (DMSO, 10 mL) was added dropwise over a period of 2 min. The mixture was stirred 2 hours at room temperature. The product was purified by dialysis against PBS (buffer volume: 20,000-50,000, concentration: 10 mM, pH=7.4) 12-24 hours with buffer solution (buffer volume: 20,000-50,000 times) changed every 3-4 hours using snake dialysis tube (MWCO: 10,000. from Pierce Inc.). The resulting solutions were combined and concentrated by ultrafiltration/centrifuged using a membrane based tube (MWCO: 30K) to yield 97 mL of the expected product with O.D. at 280 nm (2.0).

B. p97—SH

Materials: Deacetylation solution: 0.35 g of Hydroxylamine-HCl and 0.073 g of EDTA in 8 mL of 62.5 mM Sodium Phosphate, pH 7.5 buffer. Readjust the pH to 7.5 with NaOH and bring to a final volume of 10 mL. Final concentration of this buffer is 50 mM Sodium Phosphate, 25 mM EDTA, 0.5 M Hydroxylamine, and pH 7.5.

Reaction: Combine 0.4 mL of the pooled p97 fractions with 40 μl of deacetylation solution in eppendorf tube, and allow to react for 2 hours at room temperature. The product, p97-SH, is used directly without further purification to couple with activated linker-drug compounds.

C. Adriamycin-SMCC (Adr-SMCC)

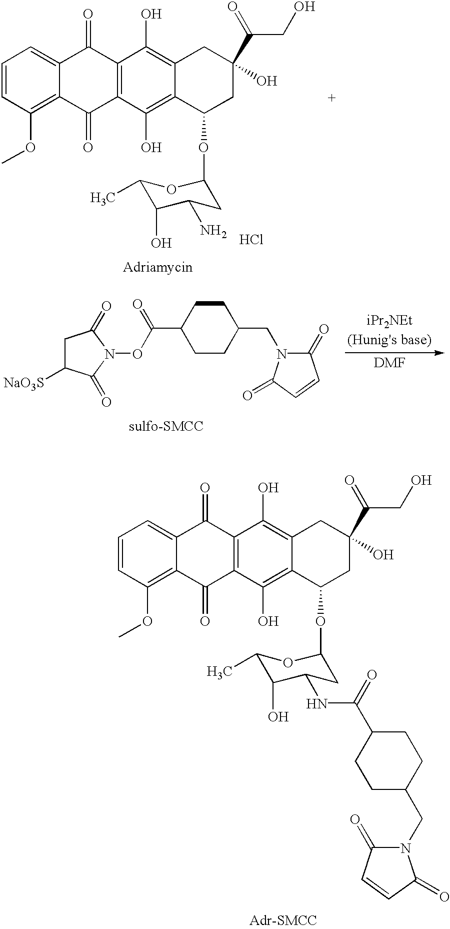

To a 250-mL, round-bottom bottom flask containing a magnetic stirrer bar was placed anhydrous DMF (150 mL, Aldrich lot #BI 01060AI), adriamycin HCl salt (795 mg, 1.371 mmol, AMRI lot #MDZ-D-95-A), sulfosuccinimidyl 4-N-maleimidomethylcyclohexane-1-carboxylate (SMCC, 550 mg, 1.645 mmol, Toronto Research Chemicals, Inc. lot#9-YCX-154-1), and diisopropylethylamine (0.36 mL, 2.057 mmol, Acros lot #AO13862501). The reaction flask was wrapped with aluminum foil and the mixture was stirred at room temperature overnight under nitrogen followed by partitioning between water (300 mL) and 10% z-propanol/EtOAC acetate (400 mL). The aqueous layer was separated and back-extracted with 10z-propanol/EtOAc (400 mL). The organic solutions were combined, subsequently washed with saturated aqueous sodium chloride (2×800 mL), and water (800 mL), followed by drying over anhydrous magnesium sulfate. The solids were filtered, the filter cake was washed with 10% z-propanol/EtOAc (3×50 mL), and the filtrate and washings were combined and concentrated under vacuum until a viscous oil was obtained. The residue was purified by silica gel chromatography eluting with 0-5% methanol/methylene chloride to yield a red solid (998 mg, 96%); mp 162-172° C.; [α]²⁵ _{D}+189.2°(c 0.12, CH₂Cl₂); ESI MS m/z 761 [C₃₉H₄₂N₂O₁₄H]⁻Rf 0.31 (94:6 CH₂Cl₂/MeOH); UV (CH₂Cl₂λₘₐₓ 202.5, 233.5, 252.0, 288.5, 478.5, 495.0 nm; ¹H NMR (300 MHz, DMSO-d₆) δ0.83 (m, 2H), 1.19 (m, 4H), 1.63 (m, 4H), 1.84 (m, 1H), 2.18 (m, 3H), 2.92 (d, J=12 Hz, 2H), 3.19 (d, J=6.8 Hz, 2H), 3.92 (m, 1H), 3.97 (s, 3H), 4.14 (q, J−6.6 Hz, 1H), 4.57 (s, 2H), 4.77 (d, J=5.6 Hz, 1H), 4.91 (m, 1H), 5.21 (s, 1H), 5.44 (s, 1H), 54.77 (s, 1H), 6.99 (s, 2H), 7.40 (d, J=8.0 Hz, 1H), 7.62 (dd, J=3.3, 3.4 Hz, 1H), 7.90 (m, 2H), 13.26 (s, 1H), 13.99 (s, 1H); ¹³CNMR (75 MHz, DMSO-d₆) δ16.9, 28.3, 28.5, 29.3, 29.6, 3.19, 36.0, 36.3, 43.0, 43.4, 44.6, 54.8, 56.5, 63.6, 66.7, 68.0, 69.9, 74.9, 100.4, 110.5, 110.7, 118.9, 119.6, 119.9, 133.9, 134.2, 134.6, 135.4, 136.1, 154.4, 156.0, 160.7, 171.1, 174.2, 186.4, 213.6; Anal. Calcd. For C₃₉H₄₂N₂O₁₄.0.5CH₂Cl₂: C, 58.92; H, 5.38; N, 3.48. Found C, 59.14; H, 5.49, N, 3.26.

D. Adriamycin-TAA Ammonium Salt (Adr-TAA Ammonium Salt)

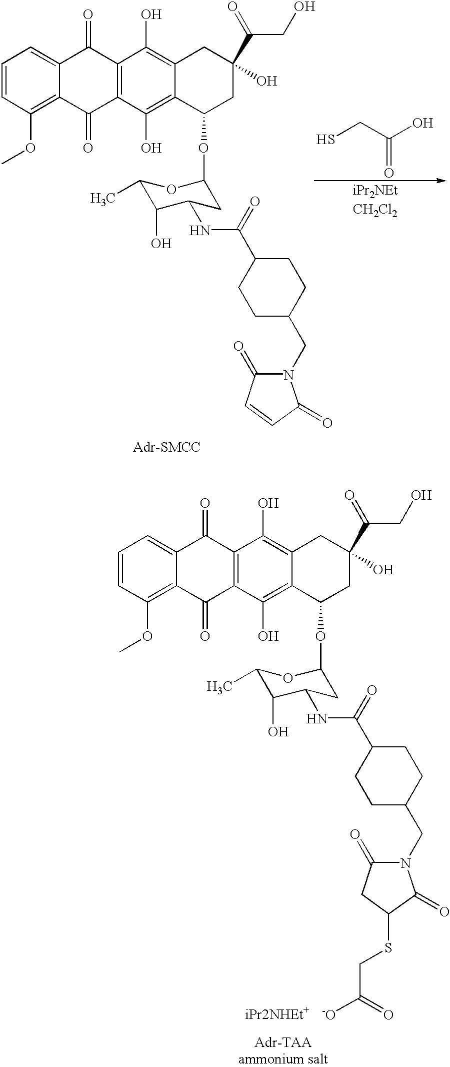

To a 500-mL, round-bottom flask containing a magnetic stirrer bar was added CH₂Cl₂ (300 mL,), ADR-SMCC (980 mg, 1.285 mmol, AMRI lot #JZ-G-30), Hunig's base (0.34 mL, 1.927 mmol, Acros lot #A013862501), and mercaptoacetic acid (0.14 mL, 1.927 mmol, Acos lot #A014921501). The reaction flask was wrapped with aluminum foil and the mixture was stirred at room temperature for 2 h under nitrogen, then concentrated under vacuum. The residue was taken up in methanol (30 mL) and the resulting methanol solution was reconcentrated. Precipitation occurred during the concentration. However, the slurry was further concentrated until about 3 mL of total volume remained. The solid was collected by vacuum filtration and washed with 10% z-PrOH/EtOAc (3×3 mL) to yield 1.178 g (96%) of the ammonium salt as a rod solid: mp 141-150° C.; [α]²⁵_{D}+218.9° (c, 0.25, MeOH/EtOAc, 2/1 v/v); ESI MS m/z 853 [C₄₉H₆₅N₃O₁₆S—C₈H₁₉N—H]⁻; Rf 0.43 (88:10:2 CH₂Cl₂/MeOH/AcOH); UV (CH₂Cl₂) λₘₐₓ 234.0, 251.6, 288.0, 400.6, 477.0 nm; ¹H NMR (300 MHz, DMSO-d₆) δ0.87 (m, 2H), 1.11-1.23 (m, 19H), 1.37-1.65 (m, 6H), 1.84 (m, 1H), 2.05-2.25 (m, 3H), 2.60 (d, J=3.5 Hz, 1H), 2.84 3.05 (m, 3H), 3.10-3.20 (m, 3H), 3.35-3.55 (m, 4H), 3.95 (m, 4H), 4.05 (m, 1H), 4.16 (q, J=6.6 Hz, 1H), 4.57 (s, 2H), 4.85 (m, 1H), 5.21 (s, 1H), 5.45 (br, 1H), 7.40 (d, J=8.0 Hz, 1H), 7.62 (dd, J=3.3, 3.4 Hz, 1H), 7.86 (m, 2H), 13.5 (br, 1H), 13.99 (s, 1H); ¹³C NMR(75 MHz, DMSO-d₆) δ17.4; 18.6, 28.8, 28.9, 29.7, 33.8, 35.7, 43.8, 44.4, 45.1, 48.9, 56.9, 64.0, 67.1, 68.6, 70.3, 75.3, 100.8, 100.9, 111.1, 119.3, 120.1, 120.3, 134.9, 135.8, 136.5, 154.8, 156.4, 161.1, 171.0, 174.6, 175.7, 177.1, 186.7. 186.8, 214.1 Anal. Calcd for C₄₉H₆₃N₃O₁₆S.3H₂O; C, 56.69, H, 6.89; N, 4.05. Found: C, 56.32: H, 6:09; N, 3.37.

E. Adriamycin-TAA Free Acid

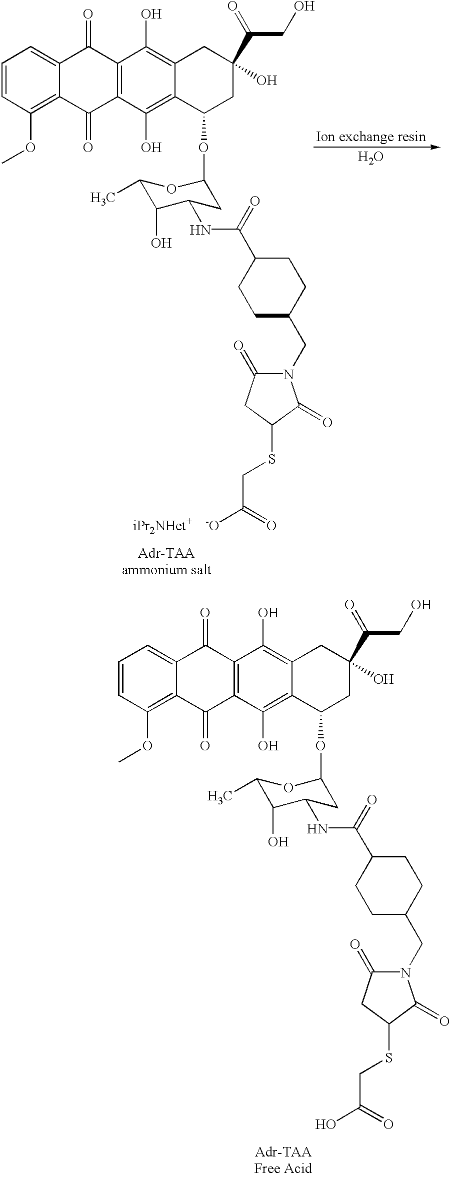

A sample of the ADR-TAA ammonium salt (242 mg, AMRI lot #JZ-G-35) was dissolved in MeOH (2 mL). The solution was loaded onto Dowex weakly acidic ion-exchange resin (8.34 g, Aldrich lot #CU 15418 PS), pre-packed in a column (1.6×32 cm) and eluted with water. The red aqueous solution was extracted with CH₂Cl₂ (3×60 mL). The extracts were combined and dried over Na₂,SO₄. The solids were vacuum filtered, and the filter cake was washed with CH₂Cl₂ (3×10 mL). The filtrate and washings were combined and the solvent was completely removed under vacuum to afford 150 mg (71%) of the free acid as a red solid: mp 143-150° C.; [α]¹²⁵_{D} +202.5° (c, 0.12, MeOH/CH₂Cl₂, 1/1 v/v); ESI MS m/z 877 [C₄₁H₄₆O₁₆S+Na] Rf 0.29 (88:10:2 CH₂Cl₂/McOH/AcOH); UV (MeOH/CH₂Cl₂, 1/1 v/v)λₘₐₓ 203.5, 234.0, 252.5, 286.5, 478.5, 495.5, 529.5 nm; ¹H NMR (300 MHz DMSO-d₆) δ0.86 (m, 2H), 1.18 (m, 5H) 1.35-1.65 (m, 1H), 1.61-1.67 (m, 3H), 1.85 (m, 1H), 2.00-2.25 (m, 3H), 2.50 (m, 2H), 2.90 (q, J=12.5 Hz, 2H), 3.17-3.20 (m, 31H), 3.38-3.46 (m, 3H), 3.62 (d, J−15 Hz, 1H), 3.96 (s, 4H), 4.05 (s, 1H), 4.14 (s, 1H), 4.58 (s, 2H), 4.62 (s, 1H), 4.75 (s, 1H), 4.91 (s, 1H), 5.21 (s, 1 H), 5.35 (s, 1H) 7.28 (d, J=10 Hz, 1H), 7.58 (d, J=5 Hz, 3, 4 Hz, 1H), 7.85 (m, 2H), 12.69 (br, 1H), 13.19 (s 1H), 13.90 (s, 1H ); ¹³NMR (75 MHz, DMSO-d₆) δ16.9, 28.4, 28.5, 29.2, 32.6, 35.2, 35.3, 43.4, 44.0, 44.7, 56.5, 63.6, 66.7, 68.1, 69.8, 74.9, 100.3, 110.5, 110.6, 119.6, 119.9, 133.9, 134.5, 135.4, 154.4, 155.9. 160.7, 170.5, 174.2, 175.0, 176.4, 186.2, 186.3, 213.4 Anal. Calcd for C₄₁H₄₆N₂O₁₀S: C, 57.60 H, 5.42; N, 3.28. Found: C, 57.87; H, 5.79; N, 2.87.

F. Adriamycin-Succinic Mono Acid

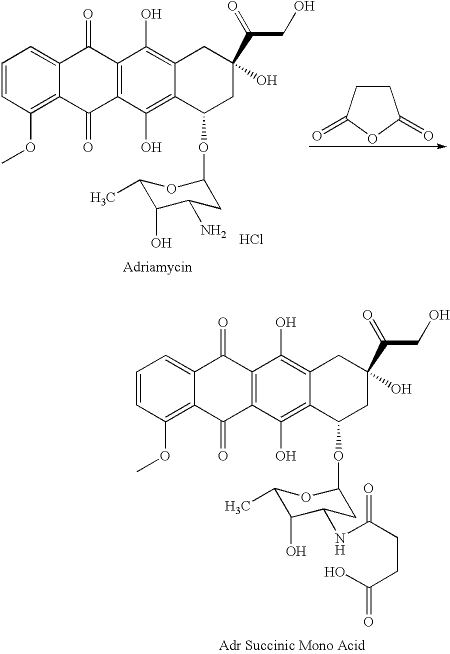

6 uL TEA (triethylamine, FW—101, d—0.726, 43.1 u mol) as added to 0.8 mL DMSO solution of 10 mg adriamycin (FW 562, 17.8 u mol). 2.7 mg of succinic anhydride (FW=100, 26.7 u mol) dissolved in 270 ul DMSO was added to the above solution. The reaction was finished within one hour at room temperature. The reaction mixture was used as is for the coupling to p97.

G. ADR-Adipic Acid Dihydrazide (Adr-ADD)

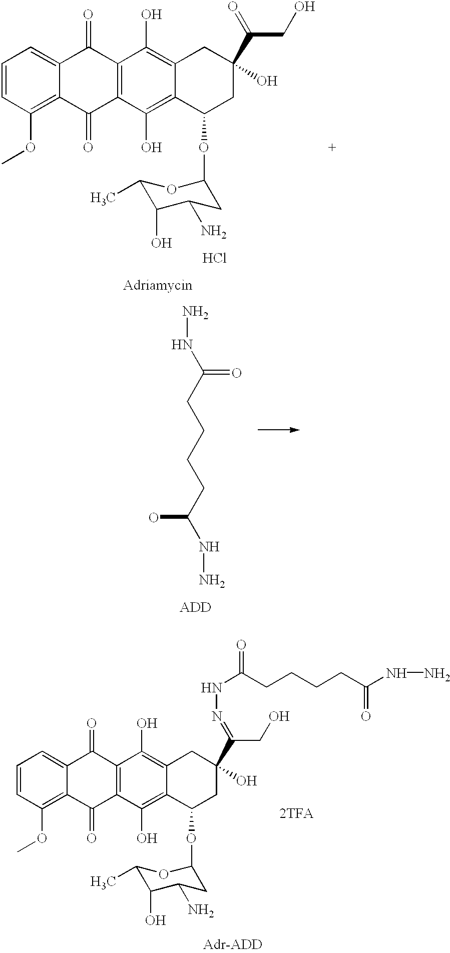

A 250-mL three-necked round-bottomed flask equipped with a nitrogen inlet and a magnetic stirrer was charged with adriamycin (580 mg, 1 mmol), adipic acid dihydrazine (190 mg, 1.09 mmol, 1.1 equiv.), and methanol (100 mL). The suspension was stirred and the nitrogen was bubbled through the solution. After 30 min, trifluoroacetic acid (0.1 mL) was introduced by a microsyringe. The reaction was monitored by TLC (dichloromethane/methanol/acetic acid, 6/3/1, v/v/v). After 5 h, the reaction was observed completed (the product Adr-ADD has R_{f}<0.1, and that of adriamycin is 0.5). The solvent was removed under vacuum at room temperature. The residue was mixed with a small amount of methanol (1 mL), and sonified to assist the partial dissolution of the solid. To this red mixture, acetonitrile was added to precipitate the product. The expected product was collected by suction filtration to yield the title product as di-trifluoroacetic acid salt (903 mg, 97%). ¹H-NMR (DMSO-d₆, 400 MHz) shows the product contains two isomers—cis- and trans- at the C—N bond. LISMS, m/z=700 (M⁺).

H. ADR-Glutaric-Mono-N-Hydroxy Succinimide Ester (Adr-Glutaric-Mono-NHS)

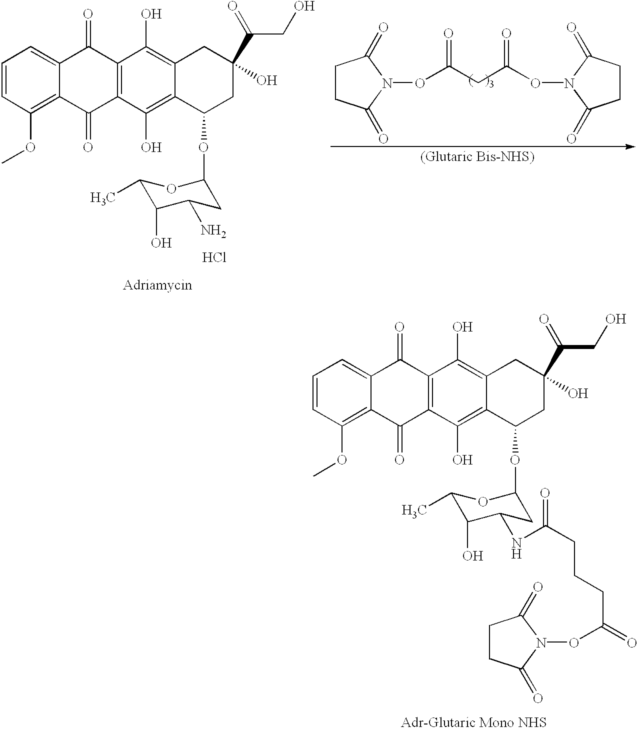

(i) Glutaric Bis-NHS Ester

A solution of glutaric acid (1 g) in DMF was stirred with NHS (2.4 equiv.) and DCC (4.0 equiv) at room temperature overnight. Simple workup and crystallization in a mixture of ethyl acetate-heptane afforded 167 g (72% yield) of the desired bis-NHS ester, the structure was confirmed by ¹H NMR and MS analysis.

(ii) Adr-Glutaric-Mono-NHS

Adr-glutaric-mono-NHS was prepared by reacting adriamycin with glutaric bis NHS ester. The structure was confirmed by ¹H NMR, ¹³C IR UV, Elemental and MS analysis.

L. Adriamycin-Adipic-Mono-NHS(Adr-Adipic-Mono-NHS)

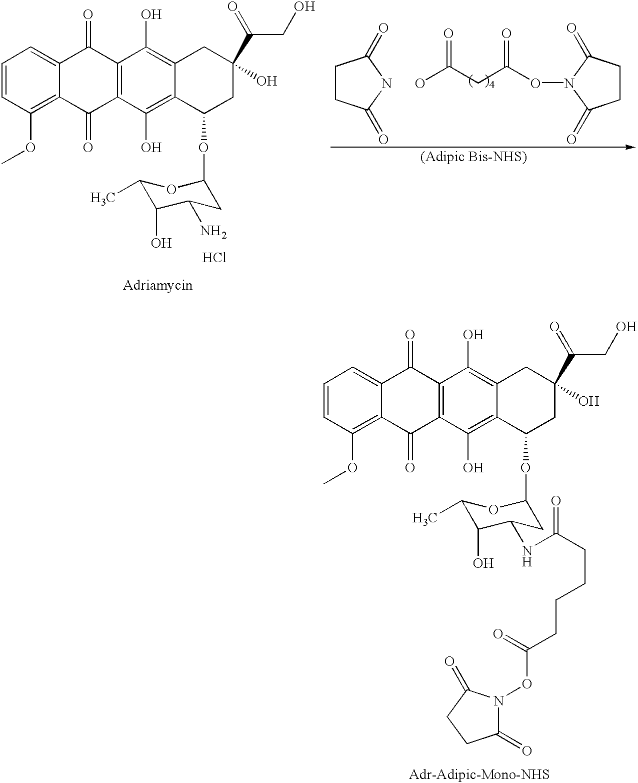

Adr-adipic-mono-NHS was prepared by reacting adriamycin with adipic-bis-NHS ester. The structure was confirmed by ¹H NMR, ¹³C NMR, IR UV, Elemental and MS analysis

J. Adriamycin-MPH(Adr-MPH)

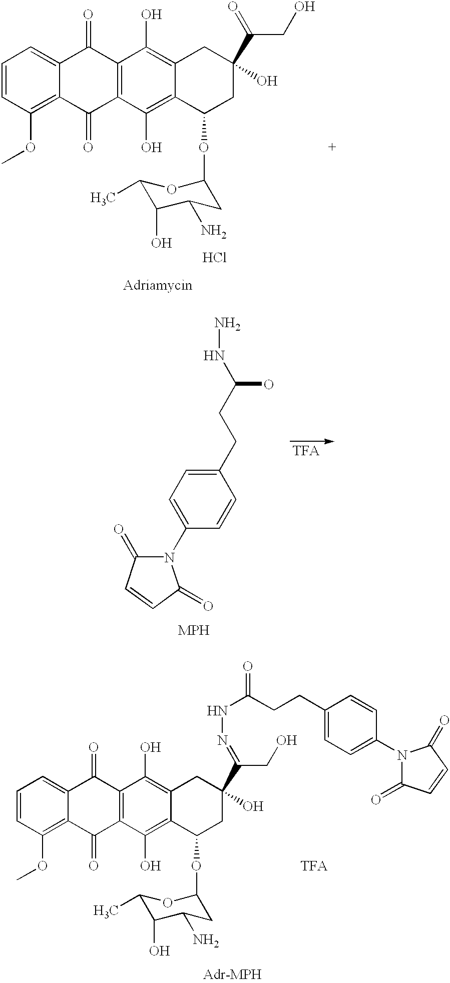

Adriamycin hydrochloride salt (580 mg, 1 mmol) was dissolved in DMSO (20 mL), then anhydrous methanol (100 mL) was added. The mixture was stirred under nitrogen for 30 min, then 4-(4-N-maleimidophenyl)butyric acid hydrazide hydrochloride ½ dioxane (MPH, 350 mg, 1.1 mmol, 1.1 equiv) was added, followed by trifluoroacetic acid (TFA, 150 uL,=1.9 mmol, 1.9 equiv). The reaction was monitored by TLC (DCM/MeOH/AcOH, 5/3/2, v/v/v). After 5 hours the reaction is completed (no more starting material converted into the product), methanol was removed under vacuum. The DMSO solution was then brought to precipitate by dropwise addition of acetonitrile, which offered 765 mg of red solid with 10-20% free adriamycin (By TLC).

K. Z′-Monosuccinoyl Taxol

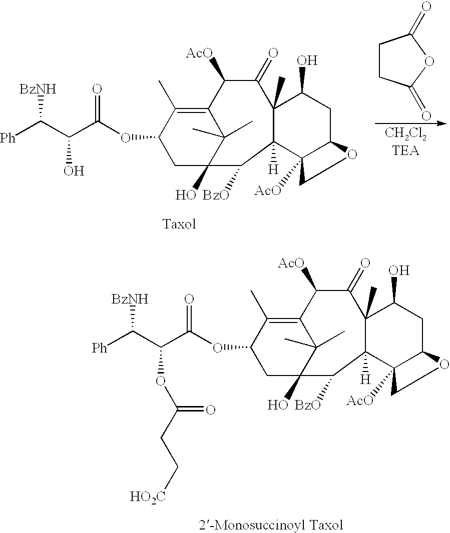

A 100-mL round-bottomed flask equipped with a magnetic stirrer was charged with taxol (300 mg, 0.351 mmol) dichloromethane (60 mL), and succinic anhydride (300 mg, 3 mmol 8.54 equiv.). The suspension was stirred and triethylamine (200 uL. 1.43 mmol, 4 equiv) was added. The reaction was monitored by TLC (dichloromethane/methanol, 95/5, v/v), and was finished after 2 h. The solution was passed through a flash silica gel column eluted with dichloromethane-methanol (95/5. v/v). After collecting the suitable band, and removing the solvent under vacuum, the residue was crystallized from dichloromethane-hexane to yield the expected product as white needle crystals (300 mg, 39%). M.p. 175-177 ° C. (lit.¹⁵ 178-180° C.). ¹H NMR (CDCl₃, 500 MHz), δ=1.5 (s, 3H, 17—CH₃), 1.20 (s, 3H, 16—CH₃), 1.65 (s, 3H, 19—CH₃), 1.88 (s, 3H, 18—CH₃), 2.20 (s, 3H, 10—OAc), 2.15, 2.30 (mm, 2H, 14—CH₂), 2.40(s, 3H, 4—OAc), 2.50-2.70 (m, 6H, 6—CH₂, 2′—OOCCH₂CH₂COOH)H 3.80 (d, 1H, J=7.02 Hz, 3—CH), 4.20, 4.30 (dd, J₁=8.42 Hz, J₂=44.33 Hz, 20—CH₂), 4.45(dd, J₁=6.70 Hz, J₂=10.88 Hz, 1H, 7—CH), 4.95(d, J=7.86 Hz, 2′—CH), 5.50(d, J−3.35 Hz, 1H, 5—CH), 5.95(d, J=3.15 Hz, 1H, 2—CH), 5.98(d, J−3.26 Hz, 1H, 3′—CH), 6.20 (t, J=8.91 Hz, 1H, 13—CH), 6.28(s, 1H, 10—CH), 7.05(d, J=9.25 Hz, 1H, NH), 7.30-8.20(m, 15H, 3 phenyl) ppm. MS (LISMS, Matrix: thioglycerol), m/e=954 (M⁺) , 654 (Taxol), 569, 509, 386.

### Example 5b

### Conjugation Reactions

A. General Procedure

p97 (120 mL, FWₚ₉₇−94420, c−1.218 mL/mg, O.D. at 280 nm 1.8, c=1.478 mg/mL, 177.3 mg=1.88×10⁻³ mmol), or p97-SH, was placed in a 250-mL round-bottomed flask equipped with a magnetic stirrer bar. The solution was cooled to 4° C. using an ice-salt bath. A solution of drug-linker compound (0.094 mmol, 50 molar equivalent of p97) in DMSO or DMF (depending on structure of drug-linkers, if it is the free acid, the compound needs to be activated, for example using benzotriaole-tetramethyluronium boron tetrafluoride). The volume of DMSO or DMF was calculated to be 15 35% based on the whole volume the reaction mixture. The ice-water bath was then removed. The mixture was stirred at room temperature for 2-24 hours. The drug-p97 conjugate is purified by dialysis against PBS (buffer volume: 20,000 50,000: concentration: 10 mM, pH=7.4) 12-24 hours with buffer solution (buffer volume: 20,000-50,000 times) changed every 3-4 hours using snake dialysis tube (MWCO; 10,000, from Pierce Inc.), MSR was measured by UV-vis method (absorption at 280, 477, and 780 nm). The purity of the conjugate was checked by FPLC (AKTA Purifer™, software UNICORN™, version 3.10 by Amersham Pharmacia Biotech) using Mono Q^{R}HR 10/10 ion exchange column, and buffer A: Tris-HCl (20 mM, pH=7.5), and buffer B: Tris-HCl/NaCl (tris: 20 mM; NaCl: 1M) as mobile phases, or using BIOSEP™ size exclusion column (From Phenomenex, Inc) and sodium phosphate buffer (10 mM, Ph=6.8) as mobile phase.

B. Adr-SMCC-S-p97

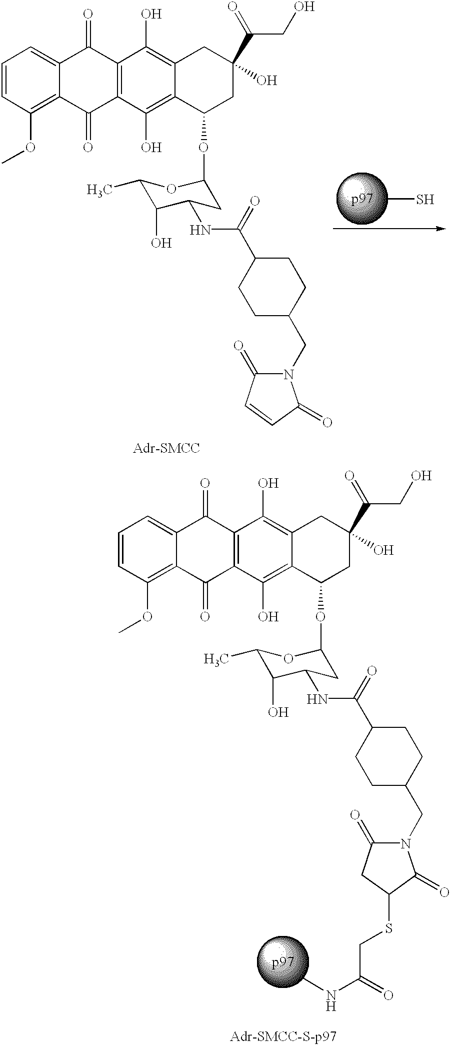

Adr-SMCC (Example 5a, C, Albany#3959, 10.5 mg, 0.0014 mmol) was dissolved in anhydrous DMSO (8.82 mL). This solution was then added dropwise into activated p97-SH (p9/-SATA) (Example 5a A, Lot#13, conc. 1.29 mL, 50 mL, −64.5 mg=0.00069 mmol). The mixture was stirred 20 hours and was then purified by column using PBS as eluent. MSR for fraction 5 is 7.85 with 49% protein recovery.

Note that in some further experiments p97adr was further labelled with I¹²⁵. This was achieved by using the p97-Adr generated in this example in a simple chloramine T reaction as described above.

C. Adr-Succinic-p97

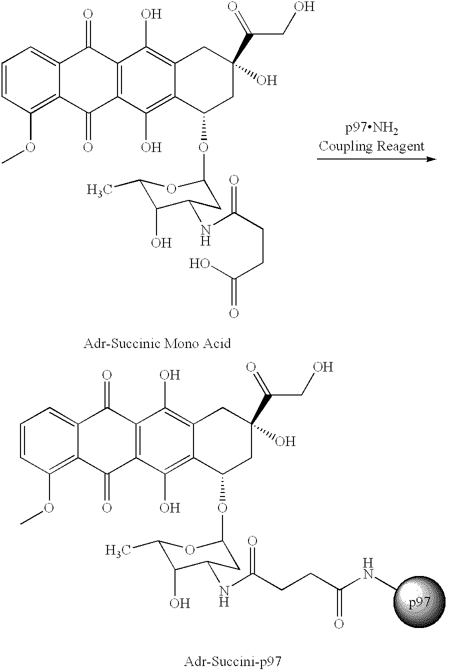

6 μL TEA (triethylamine, FW=101, d=0.726, 43.1 umol) was added to the reaction mixture from Example 5a F. 11.6 mg O Benztriazol-1-yl- N,N,N′N′-tetramethyluronium tetrafluoroborate (FW=321, 36.1 u mol) dissolved in 580 uL was then added. After one hour at room temperature, the product mixture was dropwise added to 27 mL p97 (Lot#9, O.D. 1.6, 1.31 mg/mL, p97/Adr=1/47). The reaction was run at 4° C. for 20 hours. Then the final product mixture was purified by D-salt size-exclusion column. MSR-11.

D. Adr-SMCC-S-p97

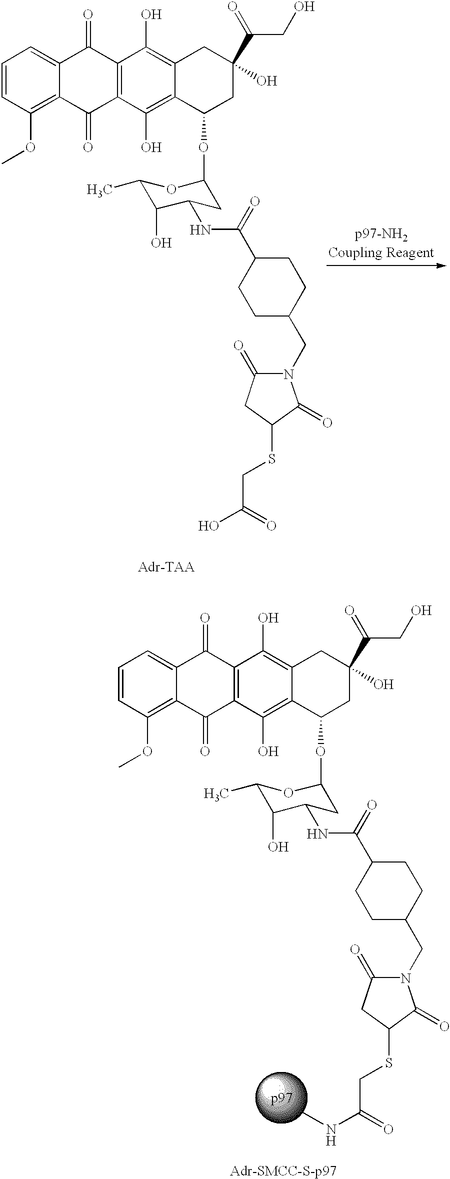

Protocol for the synthesis of 300 ml of one-step ADR-p97 conjugate with MSR=9 using Adr-TAA intermediate

Suspend ADR (150 mg) in dry DMF (6.20 mL) and start stirring in the dark at room temperature. Add triethylamine (74.9 μL) to the stirring reaction mixture. Stir for 1 h at room temperature in the dark.

In a sample tube, dissolve SMCC (116.5 mg) in dry DMF (3 mL) add the SMCC solution to the reaction mixture. Stir for 2 h10 in the dark at room temperature. Add the mercapto acetic acid (18.55 μL). Continue stirring for 2 h30 in the dark at room temperature. Add TBTU (O-benzotriazol-1-yl-N,N,N′,N′-tetramethyluronium tetrafluoroborate, 85.7 mg) to the stirring reaction mixture. Continue the stirring for 1 h.

Transfer p97 into an autoclaved round bottom flask with a strong magnetic stirrer, and cover with foil. Slowly add the ADR reaction mixture by very small aliquots to the stirring p97 solution, trying to avoid local excess of ADR in the p97 solution. When the addition is complete, rinse the ADR flask with DMF (1 mL) and add slowly to the p97 mixture. Continue stirring of the conjugate reaction mixture for 15 hours. Using 300 desalting columns previously rinsed with PBS, aliquot 1 mL of the reaction mixture on each column and elute into “fraction 1” sample tube until the solution has flowed completely into the column. Add PBS (0.5 mL) onto the columns and elute in “fraction 2” bottle until the solution has flowed completely into the column. Add PBS (0.5 mL) onto the columns and elute in “fraction 3” bottle until the solution has flowed completely into the column. Add successively PBS; 0.5 mL and 0.5 mL and elute each time in “fraction 4” bottle until the solution has flowed completely into the column. Add PBS (0.25 mL) onto the columns and elute in “fraction 5” bottle until the solution has flowed completely into the column. Check the OD and MSR of fractions #3, 4, and 5 and join the fractions if the MSR are similar.

E. Adr-ADD-p97SATA

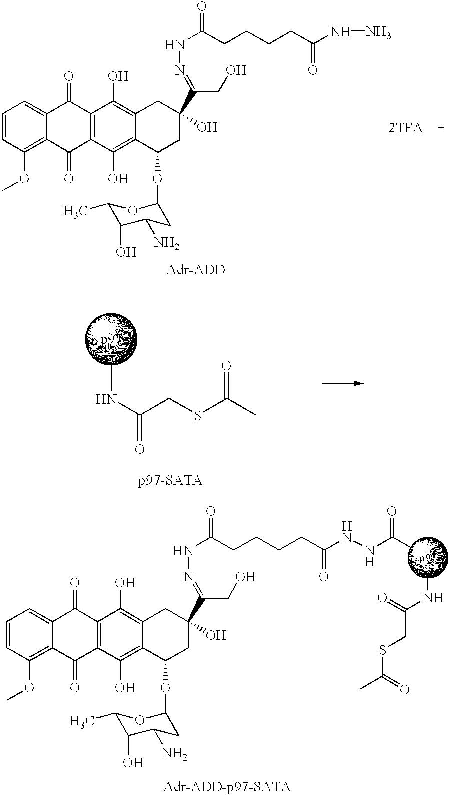

A 100-mL round-bottomed flask equipped with magnetic stirrer was charged with p97-SATA (See Example 5a, A, Lot#QC2P40, 50 mL, O.D.=2.0/280 nm, ˜8.4e-4 mmol) and Adr-ADD (Example 5a, G, Lot#QC2P37, 39 mg, 0.04 mmol, 50 equiv to p97-SATA). The flask was placed into the ultrasonic bath for a few minutes to help Adr-ADD to dissolve. Then the mixture was stirred and 1-ethyl-3-dimethyl-3-propylamino-carbodiimide hydrochloride (EDC, 65 mg, 0.33 mmol, 400 equiv to p97-SATA), and sulfo-NHS (36.8 mg, 0.18 mmol, 200 equiv to p97-SATA) were added at once. The mixture was stirred at room temperature for 3 hr, kept overnight at 4° C., then filtered through 0.2 μm nylon membrane and purified by Dialysis using Slide-A-Lyzer 10K against 0.01 M PBS buffer, to yield ˜70 mL produce OD—0.6060/480 nm, 1.4457/280 nm. MSR=6.29.

F: Adr-Glutaric-p97

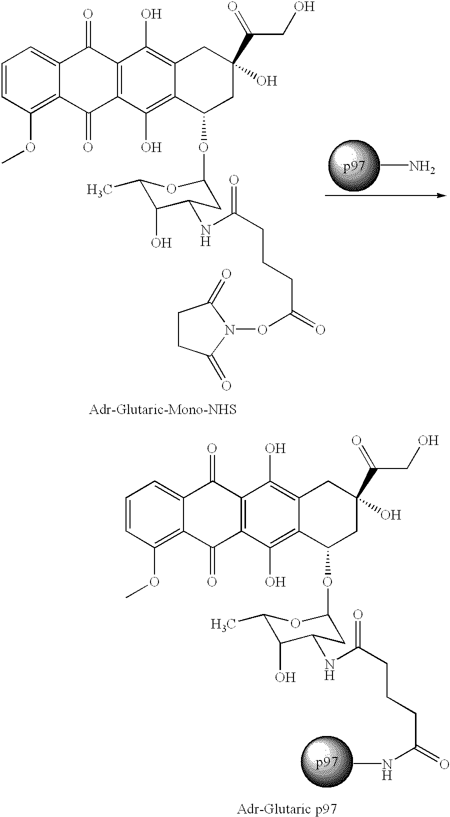

Dissolved 17.2 mg (22 umol) Adr-gutaric-mono-NHS (Example 5a H, in 2.6 mL DMSO. Combined this solution with 79 mL p⁹⁷ (lot#13a, OD—1.60, 1.1 umol). The mixture was stirred at 4 ° C. overnight. The compound was purified by D-Salt column. MSR=4.6. The compound was still stable after 40 days.

G. Adr-MPH-S-p97

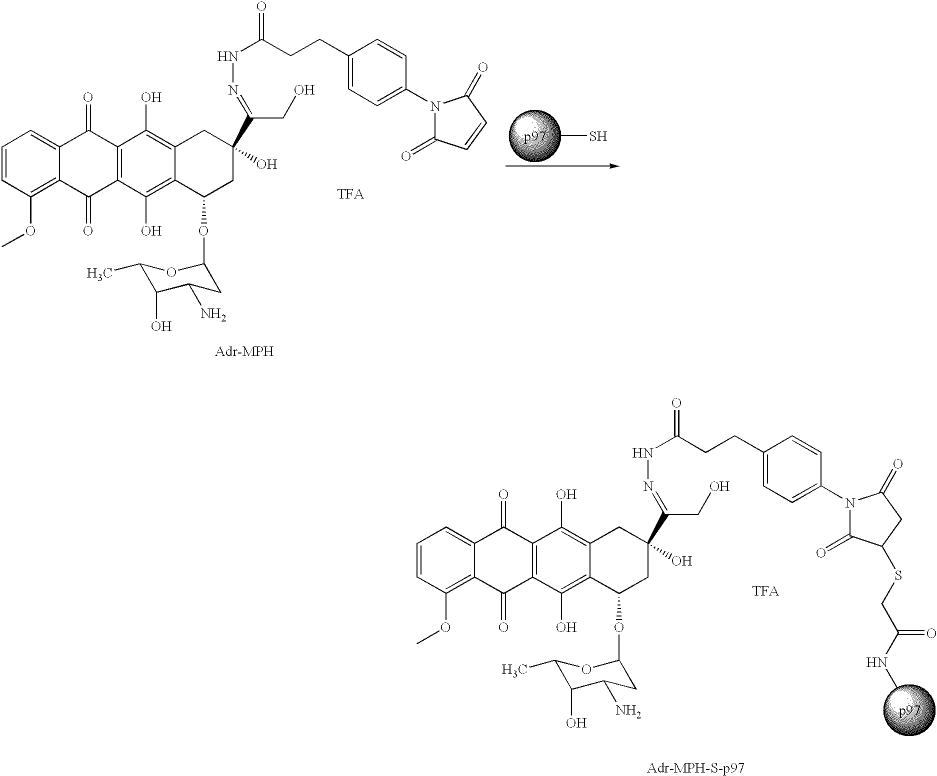

p97-SH (p97-SATA, Example 5a A, Lot QC2P48, O.D.=1.48, 47 mL, 5.9e⁻⁴ mmol) was stirred at room temperature for 30 min with hydroxamine hydrochloride salt (308 mg, 4000 equiv). Then a solution of Adr-MPH (Example 5a J, Lot QC2P12, 10 mg,˜100 equiv) in DMSO (8 mL) was added dropwise. Then the pH was adjusted to 7.4 by using aqueous saturated sodium acetate, The mixture was stirred overnight at 4 ° C. then purified by dialysis. The MSR was 5.26, and 4.41 after one filtration.

H. Adr-Adipic-p97

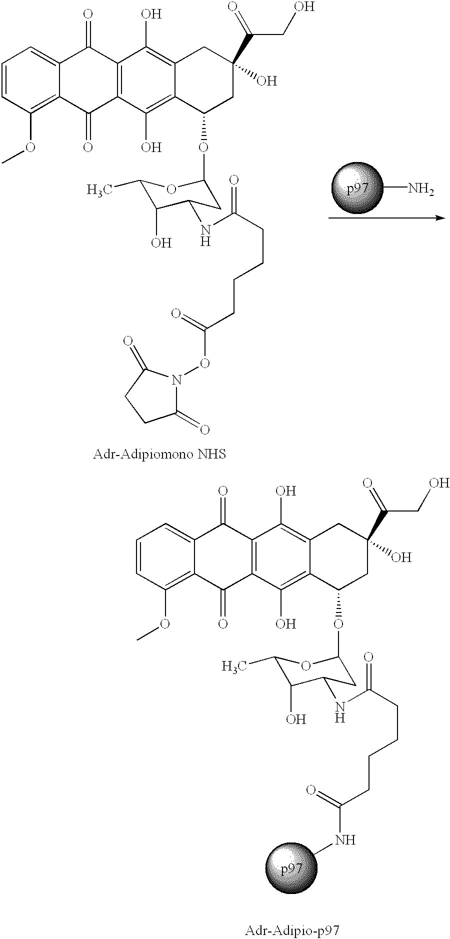

ADR-Adipic-mono-NHS (Example 5a I, 12.3 mg, 15.65 mmol) was dissolved in DMSO (2 mL). Then this solution was added dropwise into a solution of p97 (Lot#14a, O.D. 1.8, 0.7826 nmol). The mixture was stirred overnight at 4 ° C. The product was purified by ultrafiltration using 1 L PBS, then concentrated to 50 mL. MSR=9.50.

L. p-97-Taxol

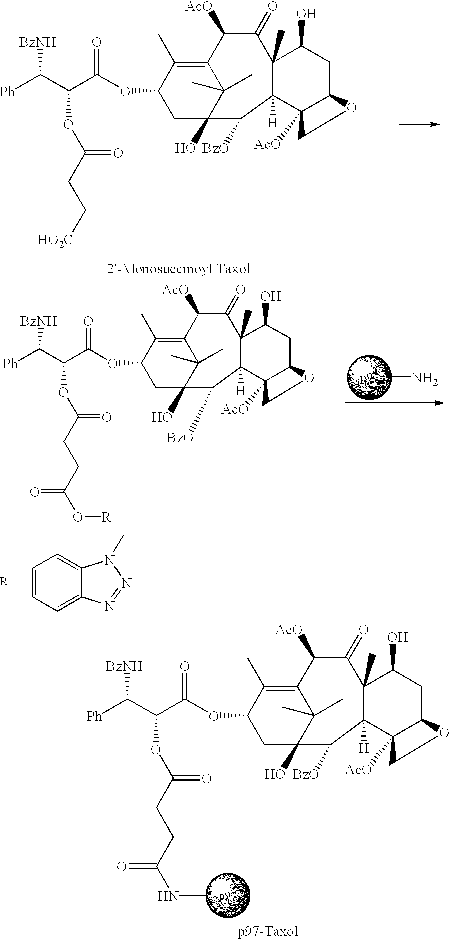

In a 100 mL round bottomed flask equipped with a magnetic stirrer was charged with 2′-monosuccinoyl taxol (66 mg, 0.069 mmol, 50 molar equivalent of p⁹⁷), benzotriazole-N,N,N′,N′-tetramethyluronyl-tetrafluoride boroate (33.3 mg, 0.104 mmol, 1.5 molar equivalent of 2′-monosuccinoyl taxol), and anhydrous dimethyl formamide (25 mL, 20% in the reaction mixture of p97). The mixture was stirred and anhydrous triethylamine (14 μL, 1.04 mmole, 150 molar equivalent of 2′-monosuccinoyl taxol) was introduced by syringe. The mixture was allowed to stir at room temperature for 60 min. TLC (dichloromethane/methanol, 95/5, v/v) showed that the starting material had disappeared and the activated carboxy-complex was formed.

In a 250 mL round-bottomed flask equipped with a magnetic stirrer p97 (100 mL, Lot@8, OD280 nm=1.6, c=1.3136 mg/mL, 1.391×10⁻³ mmol) was added. The stirrer was started, and the activated carboxy-complex prepared above was introduced by syringe over a period of 5 min. The mixture was stirred at 5° C. The conjugate was purified by Dialysis using snake tube against PBS buffer (pH=7.40) with buffer change every 4 hours and total 3 times. The expected conjugate was obtained and analysized by FPLC (BioStep™Sec-S 3000 size-exclusion column using 0.01M pH=6.8, PBS buffer, or Mono Q^{R} HR 10/10 ion exchange column using a combination of 0.01 M, pH=7.1 PBS buffer with 1M NaCl-0.001 M PBS buffers).

J. p97-Cisplatinum

Conjugation of cisplatinum to p97 may be carried out as in Example 5b, B, with replacement of adriamycin by the same concentration of cisplatinum-SMCC, prepared as in Example 5a C but changing the SMCC concentration to 84 mM.

K. p97-Horse Radish Peroxidase

Conjugation of horse radish peroxidase to p97 was carried out as in Example 5b B with replacement of adriamycin with 10 mg/mL purified horse radish peroxidase-SMCC, prepared as in Example 5a Cbut changing SMCC concentration to 10 mM. Additionally, it may be advisable to use p97 concentrations up to 4.5 mg/mL.

L. p97-Cisplatinum-adriamycin

A multiple combination p97-chemotherapeutic agent may be made using the SATA protocol of Example 5b B with the following modifications: For p97-cisplatinum-adriamycin the reaction requires the following mol:mol ratios:

CisPt:ADR=1:1

ADR:SATA=1:2

CisPt:SMCC—1:10

CisPt:p97 (at start)=45:1

Solutions were initially prepared in standard buffers as follows: CisPt=2.5 mg/mL; ADR=2.5 mg/mL; SATA=5.25 mg/mL for ADR; SATA=10 mg/mL for p97; SulfoSMCC=36.5 mg/mL (84 M); p97 -1.3 mg/mL.

For generating CisPt-ADR complex, first incubate 4×75 μl CisPt and 4×75 μl SMCC for 3.5 hours at room temp. Separately, incubate 4×145 μl ADR+4×55 μl SATA for 1.5 h at room temp. Separately incubate 4×200 μl ADR-SATA+4×50 μl deacelylation solution for 2 h at room temp. Finally, incubate 4×250 μl dADR-SATA+4×150 μl CisPt-SMCC for 1-3 hr at room temp (Compound A).

For generating p97 SATA derivative: incubate 8×500 μl p97+8×50 μl SATA for 1 h at rT. Desalt over 4 desalting columns and collect fractions 6-11 (1⁶ increments). Then incubate 16×200 μl p97-SATA+16×20 μl deacelylation solution for 3 h at RT (compound B).

Generating p97-CisPt-Adr complex, Incubate 16×220 μl dp97-SATA (Compound B)+16×100 μl CisPt-ADR (compound A) overnight at 4° C. Desalt over 4 columns.

M. p97-HYNIC

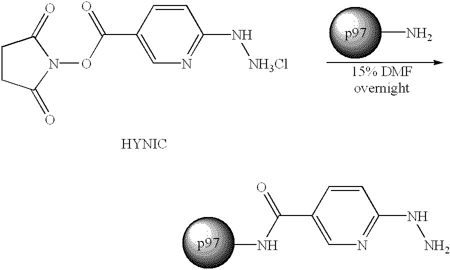

Succinimidyl hydrazino nicotinic hydrochloride (HYNIC) was synthesized according to Abrams, (J. Nuc. Med. 31:2022-2028, 1990). After this was complete, the next step is to conjugate the linker to p⁹⁷. As mole ratios, etc. can not be determine theoretically, it was decided to test a number of molar excesses of HYNIC in p97. The HYNIC was dissolved in DMF and then added to a stirring solution of p97 at 1.3 mg/ml, with total DMF kept at 15% to minimize the denaturation of the p97. Solutions were stirred overnight at 4° C. and kept shielded from light. The samples were purified by dialysis into PBS×1, pH=7.4 with 2 buffer changes in 24 hours. Analysis may be performed by a dye binding assay from Pierce, called BCA Assay. This determined the concentration of the protein in solution. HYNIC bound to the protein may be analyzed by hydrazone assay, which involves mixing the conjugate with p-nitrobenzaldehyde in a solution of 2.5% acetonitrile in 0.1M acetate buffer, pH=4.7 and letting incubate for five hours.

### Example 5c

This example describes methods of influencing linkage ratios.

According to this invention, and as described herein, investigators may seek to obtain different mol:mol ratios of p97-therapeutic agent. For some applications a 1:1 ratio may be used, for others, 1:10 or higher is used.

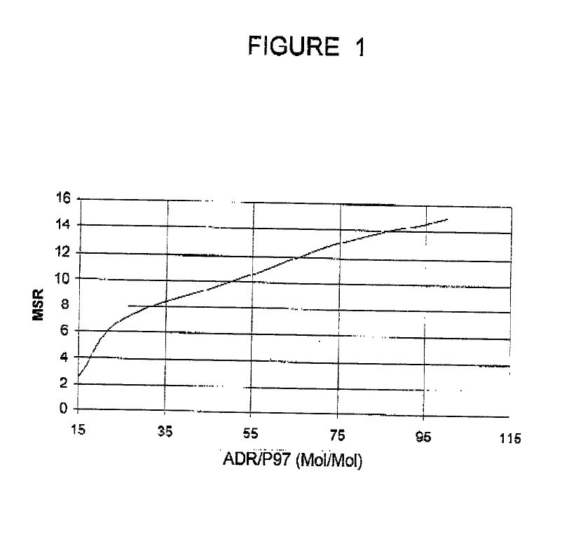

While any chemical conjugation method may be used, a preferred method employs SATA to cross react with amines in lysine residues on the protein. Theoretically, p97 has a total of 25 lysine residues. By standard colorimetric Molar Substitution Ratio (MSR) analysis, approximately 20 amine groups were found to be available for linking in solution, a number that corresponds to the expected amounts. FIG. 1 shows that by increasing the relative amount of activated ADR to activated p⁹7 in the conjugation reaction, the MSR can be increased from 1 to up to 15,and possibly higher. Thus, p97-compound ratios are tunable according to the needs and desires of the particular usage. A technique to improve linkage ratios is to purify the ADR-SMCC conjugate before linking to p97-SATA. This additional step will remove contaminants, which block free amino groups on the p97.

### Example 6

### The Influence of p97 Versus BSA for Compound Delivery

p9⁷ or BSA (bovine serum albumin, control) were prepared and iodinated with I¹²⁵ using a chloramine T protocol. Where indicated, p97- I¹²⁵ was treated according to the methods set out above to generate Apo p97- I¹²⁵ (essentially iron free p97) and Holo p97- I¹²⁵ (p97 loaded with FeCl₃). 1×10⁷ DPM of sample was prepared in 200 μl buffer (100 mM NaCl and 20 mM HCO₃) and administered to C57 black mice (( 16-20 g) by tail vein injection. At the indicated time point, mice were given an overdose of Ketamine/Xylazine anaesthetic mix. The chest was opened and blood was removed with a 27 gauge needle via cardiac puncture. The left atria was snipped open and the mouse was perfused with heparinised saline to flush out any serium associated counts from the vascular system. Then organs wore removed. I¹²⁵ counts were read directly from whole organs in a gamma scintillation counter.

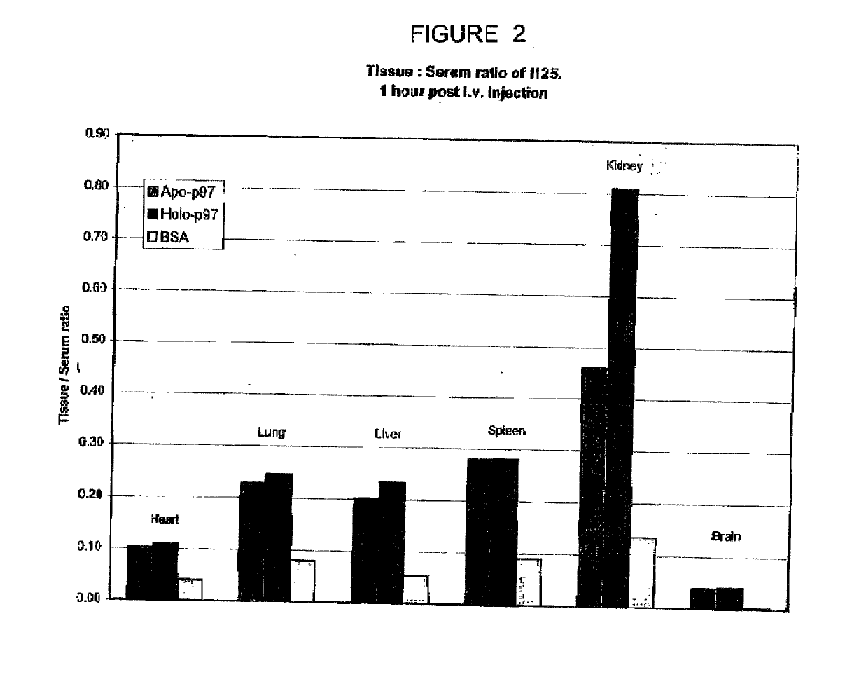

FIG. 2 shows the tissue/serum ratio at 60 minutes after injection of Apo P97-I¹²⁵, Holo p97- I¹²⁵ and BSA-I¹²⁵. Every organ, including the brain, demonstrates significantly increased uptake of p97 compared to BSA. No significant difference is identified between the Apo and Holo forms of the protein (kidney results not confirmed as significant). At 1 hour, BSA linked compounds remain in serum to a significantly higher degree than p97 linked compounds.

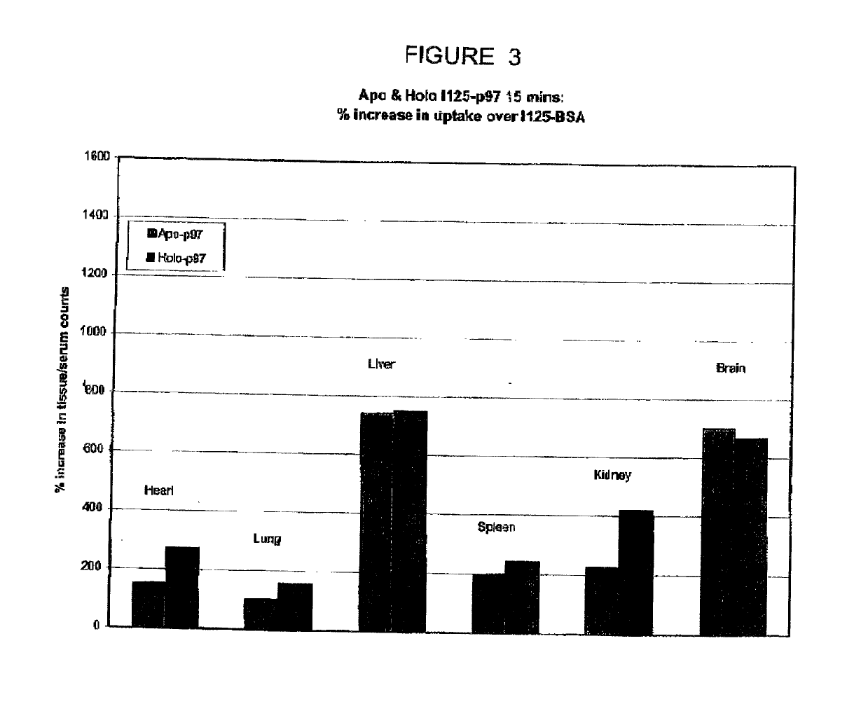

FIG. 3 shows the relative increase at 15 minutes in p97 uptake over BSA. These results indicate that p97-compounds preferentially accumulate in the brain. Again no difference between Apo and Holo forms of p97 are identified.

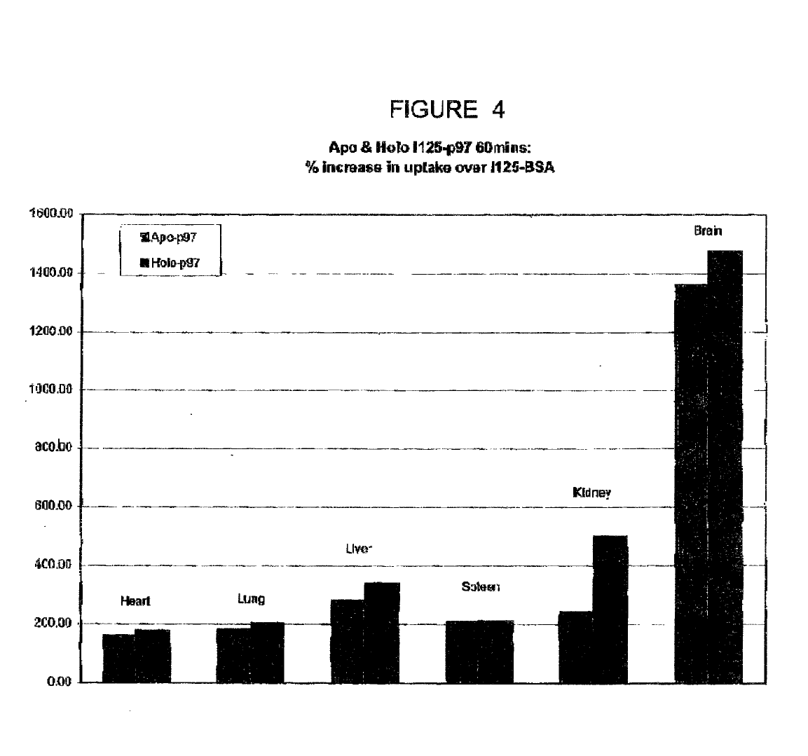

FIG. 4 shows that at 60 minutes, the brain tissue demonstrates a very significant relative increase of p97 uptake over BSA uptake (almost 15 times higher). This differential is greater for the brain than for any other organ observed in this study.

### Example 7

### In-vivo Pharmacekinetics Study in Tumour-bearing Mice

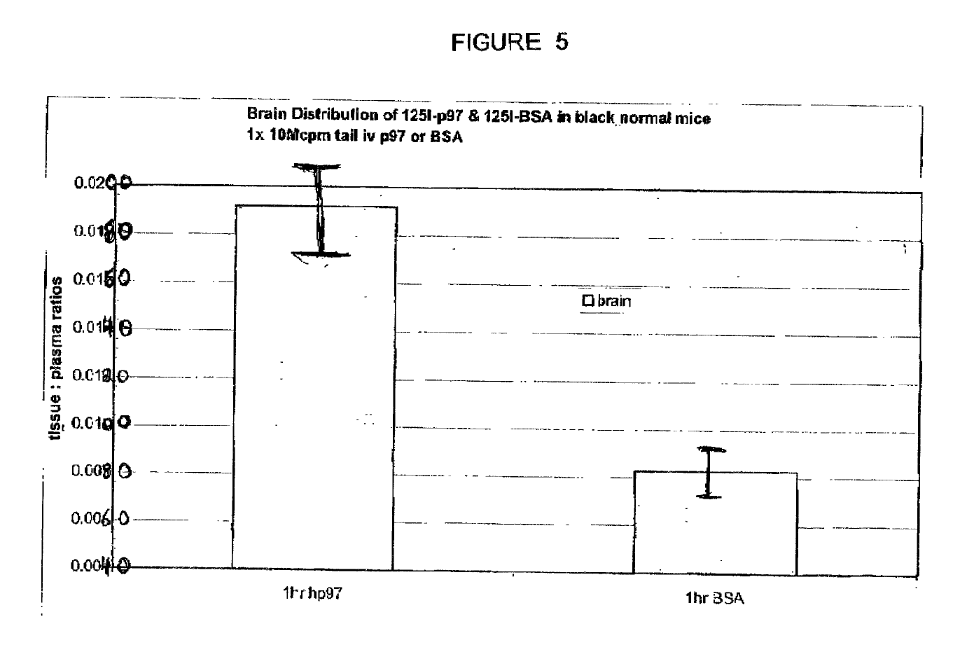

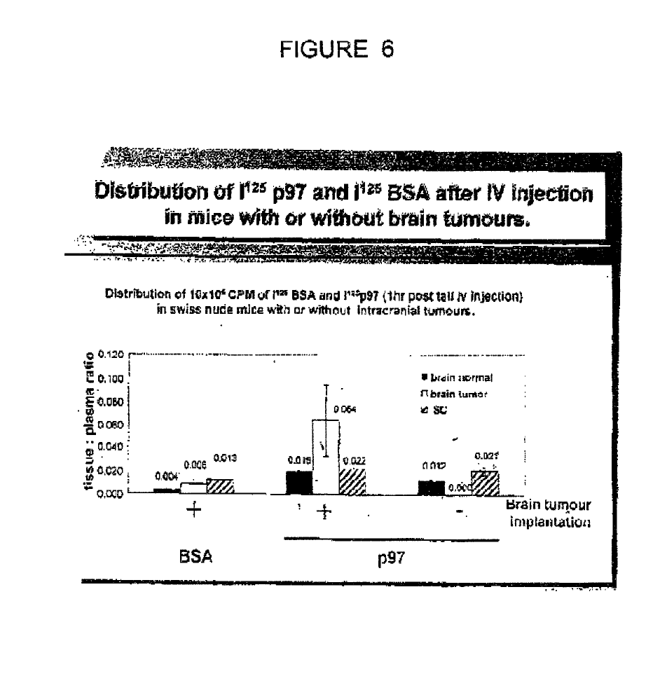

Female NSWNU(m) Swiss nu/nu aged 6-8 weeks weighing 20-30 g (Charles River Labs) were placed in micro isolated cages (5 mice per cage) in a hepa filtered ventilated animal rack under positive air pressure. They were housed for at least 1 week prior to the experiment and were allowed food and water ad libitum. In FIG. 5, normal black mice were used. In FIG. 6, Mice were implanted intracranially with 4×10⁵ C6 glioma cells, followed by a waiting period of at least 13 days for tumour growth, according to protocols elsewhere in this specification.

The compounds tested were radiolabelled BSA (bovine serum albumin, control) (EM Science) and radiolabelled p97 protein. The p97 protein is labeled with radioisotope 125-iodine using a standard chloramine T method. The labeling efficiency was >90% and checked by trichloroacetic acid precipitation. The concentration of labeled p97 was 0.32 μg/ml measured by Pandex. The specific radioactivity of p97 ranges from 230 to 360 μCi/mg. The radioactivity was measured by COBRA™ II auto-gamma counter. The p97 synthesized by Synapse was concentrated using Vivapore concentrating device and determined 10 mg/ml by OD280. BSA was also labelled with 125-iodine using the same method. BSA was dissolved in 10 mM pH7.4 PBS (phosphate buffer saline) to obtain 10 mg/ml.

Treatment: In FIG. 5, 1×10 Mcpm ¹²⁵I-p₉₇ protein, i.e., 4.32 μg (2.2 to 3.4 Mcpm) was injected into the mouse via its tail vein. An equivalent cpm dose of ¹²⁵I-BSA was injected in control animals . In FIG. 6, A fixed amount of ¹²⁵I-p97 protein, i.e., 432 μg (2.2 to 3.4 Mcpm) was injected into the tumour bearing mouse via its tail vein. An equivalent cpm dose (i.e., 2.2 to 3.4 Mcpm) of ¹²⁵I-BSA was injected in control animals. All animals were cared for under approved animal care protocols. At 1 hour after injection, animals were sacrificed with an overdose (0.10-0.15 ml) of anesthetic (1:5 xylazine:ketamine) and the indicated organs were collected. In this case there was no tissue perfusion prior to organ collection.

The results of the study are shown in FIG. 5 and FIG. 6.

FIG. 5 shows that in black normal mice ¹²⁵I p97 protein accumulates in the brain at substantially greater levels than ¹²⁵I-BSA, thus illustrating its benefits as a generalized delivery vehicle for conjugated therapeutic agents.

FIG. 6 shows several important points. Firstly, in the tumour bearing mice, p97 accumulation is greater than BSA in both brain and spinal cord, and substantially greater for accumulation in the neurological tumour. This supports the therapeutic efficacy, in neurological tumours, of p97 ADR. Additionally, the chart shows that the brain accumulation of p97 is not significantly influence in the presence of absence of the neurological tumour, which at the time of the experiment is often quite large, perhaps one third of the total brain volume. Thus these experiments confirm that the blood brain barrier remains intact in the presence of the tumour.

### Example 8

### p97 Influences Biodistribution of Compound

In the following examples, p97 is conjugated to [¹⁴C]ADR to generate p97-[¹⁴C]ADR using the techniques set out above. p97-[¹⁴C]ADR so generated was found to have specific activity of 57 mCi/mmole. A solution of 500,000 dpm/mouse of this formulation in 100 μL was injected intraperitoneally in each mouse. The same amount of free [¹⁴C]ADR was injected into comparative mice. At 1 hour after injection, mice are terminated and organs are prepared as before. Tissue is solubilized and read in a scinallation counter.

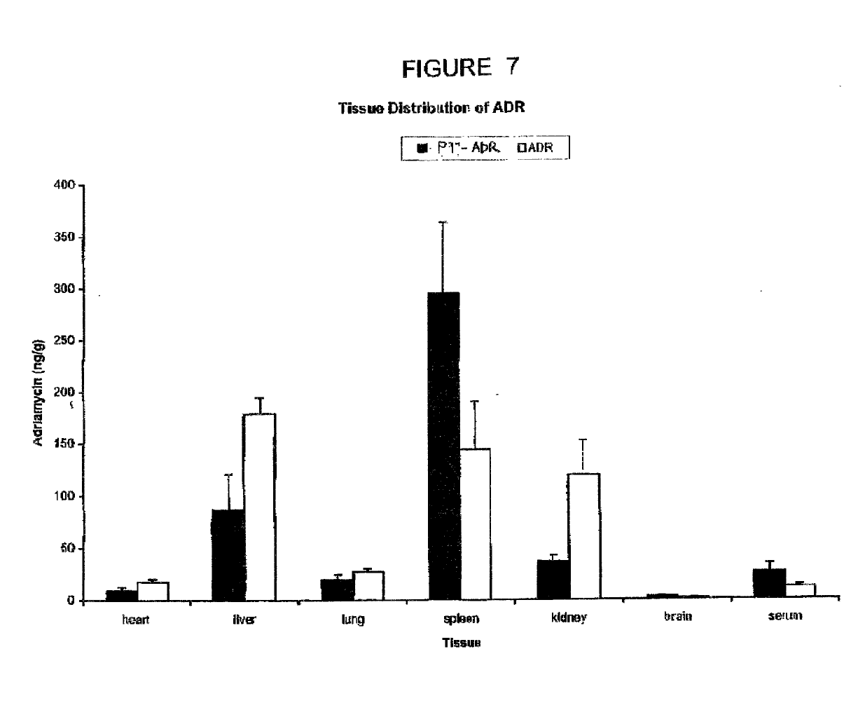

FIG. 7 illustrates accumulation at various organs of p97-ADR. The data demonstrates that ADR linked to p97 has a significantly different biodistribution than free ADR. The results demonstrate that p97 enhances delivery of ADR to spleen; and permits longer serum circulation time for the drug than free ADR. Additionally, p97 exerts a strong protective effect on the heart, liver, and kidney and reduces accumulation of ADR at those organs.

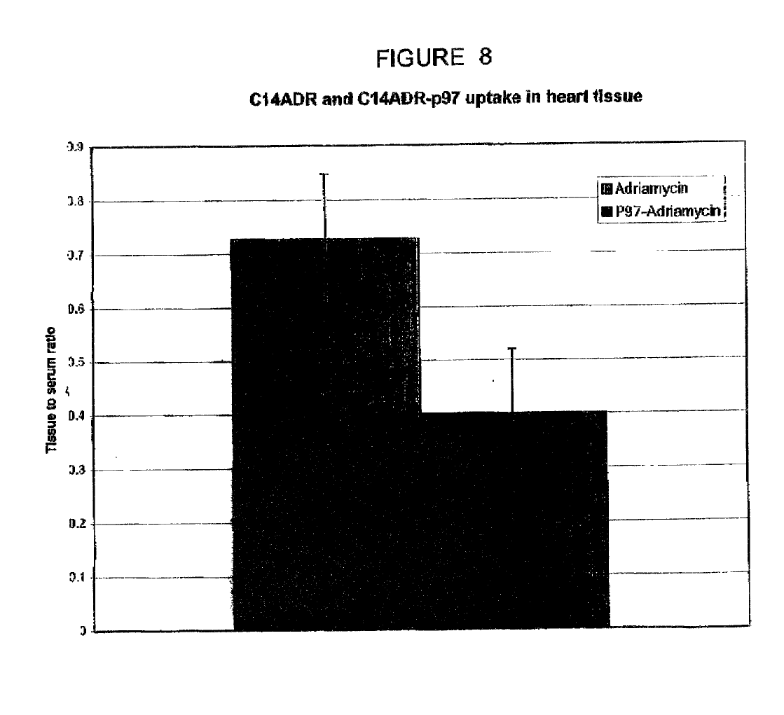

FIG. 8 illustrates the significant difference in tissue to serum ratio of heart tissue between p97-ADR and ADR. Linkage of the cardiotoxic drug ADR to p97 will significantly reduce cardiotoxicity of the dose of drug. Alternatively, compounds of this invention now permit the administration of a much higher amount of ADR than previously possible, without increasing the cardiotoxic consequences of such treatment.

### Example 9

### Reduced Cardiotoxicity of Adriamycin when in p97-ADR Conjugate

This example demonstrates that cardiotoxicity of adriamycin can be substantially reduced by administering the adriamycin as a p97-ADR conjugate.

Serum enzyme activity (CPK and LDH) was measured in mice according to standard techniques, five minutes after tail vein injection of 5 mg of adriamycin and p97-ADR conjugate (prepared according to the proceeding examples.).

| | LDH | CPK |
|---|---|---|
| free adriamycin (5 mg) | 1630 ± 84 | 773 ± 21 |
| P97-ADR (5 mg doxo) | 624 ± 41 | 198 ± 12 |
| saline | 557 ± 33 | 129 ± 78 |
| N-3/group | | |

Results show that administration of a therapeutically effective dose of adriamycin in the form of a p97-ADR conjugate substantially reduces the cardiotoxic effects of the free compound.

### Example 10

### Detection of Free ADR and Taxol in the Brain After Injection of p97-ADR and p97-Taxol

This example shows the detection of free therapeutic agent in the brain and/or neurological tumour, after intravenous delivery of 1) p97-ADR conjugate or free ADR control, and 2) p97-taxol conjugate or free taxol control. The results show that in both cases, relative to free compound, the p97-chemotherapeutic agent conjugate substantially increases delivery of the chemotherapeutic agent to the brain and neurological tumours.

This example also shows that the conjugated compound is released from the p97 and can be found in the free form in the brain and/or the neurological tumour.

Test compounds were obtained as follows: free taxol (commercial supplier); p97-taxol: Synthesis according to example 5b(I); Adriamycin (commercial supplier); p97-adriamycin was synthesized according to the general protocol.

Treatment of Mice. In each case, the test compounds and controls were administered in a 100 μl injection to mice by tail vein injection as described elsewhere in this specification. All animals were cared for under approved animal care protocols, At the indicated time points, animals were sacrificed with an overdose (0.10-0.15 ml) of anesthetic (1:5 xylazine:ketamine) and organs and tissues collected.

For the p97-taxol/taxol experiment C57BL/6 male mice (non-tumour bearing) were used.

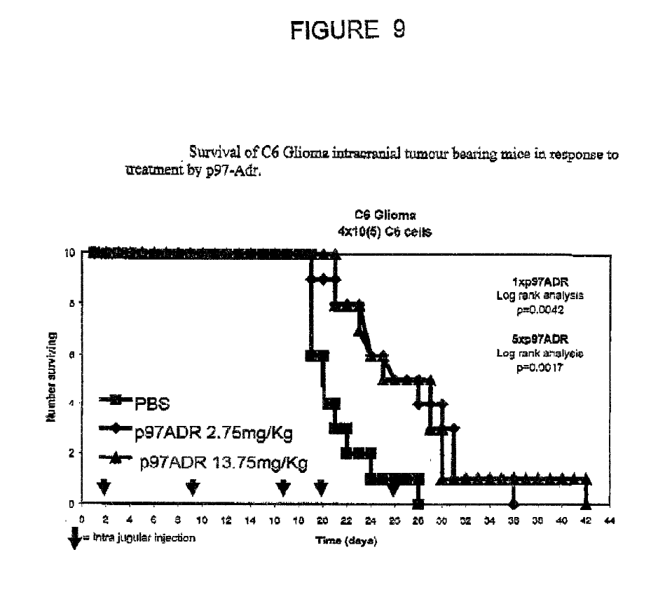

For the p97-Adr/Adr experiment, Nude female mice bearing IC tumours, according to the tumour implantation protocols set out below (4×10⁵ C6 glioma tumour cells). Mice were sacrificed 1 hour following the fifth injection of p97-ADR as set out in Example 11 FIG. 9 below.

Determination of adriamycin, taxol and metabolites in vitro by HPLC: After an incubation, the tissues were homogenized in 4% (w/v) BSA in water, resulting in final concentrations of approximately 0.05-0.2 g tissue/ml. A 200-μl aliquot of each sample was added to 200-μl of a 6% (w/v) borate buffer (pH 9.5) and 100-μl internal standard (daunorubicin). The analytes were extracted from the samples with 1 ml chloroform-1-propanol (4:1, v/v) by mixing, followed by certification for 10 min at 4° C. (3000 g). The organic, layer was evaporated by vacuum. The residue was reconstituted in 100 μl of acetonitrile-tetrahydrofuran (40:1. v/v), and 300 μl acidified water (pH 2.05). A 50 μl aliquot was injected into the HPLC system.

An analytical column Eclipse XDB-C8 (150×4.6 mm I.D.) packed with 5 μm reversed-phase was used. The mobile phase consisted of acidified water (pH 2.05)-acetonitrile-tetrahydrofuran (80:30;1, v/v/v). A flow-rate of 0.4 ml/min was used. For detection of ADR and the ADR-SMCC intermediate, the column eluent was monitored fluorometrically at an excitation wavelength of 460 nm and an emission wavelength of 550 nm.

Results are shown in Table 1 and Table 2.

In both cases, tail vein injection of p97-compound conjugate delivered substantially increased amounts of compound to the brain and/or neurological tumour compared to free compound administered the same way.

### Example 11

### Improved Survival with Treatment with p97-ADR

The mice used for this trial were female NSWNU Swiss nu/nu 5-7 weeks of age supplied by Taconic Farms Inc. All mice were housed in micro isolated cages (5 mice per cage) under positive air pressure in a Hepa filtered ventilated animal rack. C6 glioma cells (ATCC CRL—2199), were cultured in Dulbecco's modified Eagles medium (DMEM) supplemented with 10% heat inactivated calf serum. Following anesthetization with 100 mg/Kg Ketamine and 10 mg/Kg Xylazine, the mice were secured in a stereotaxic injection frame. 5 μL of sterile phosphate buffered saline (PBS) containing 4×10⁵ C6 cells was injected at a rate of 1 μL per minute 3 mm below the surface of the skull 3 mm in front of the coronal suture and 3 mm to the right of the midline. Injections were given from a 25 μL syringe with a 27 gauge needle. Injection volume and rate were controlled using a motorized injector. One minute after the end of the injection the needle was removed slowly and the injection hole sealed using sterile bone wax, and the scalp closed with sterile sutures.

In FIG. 9, efficacy of p97-ADR against C6 growth was assessed by administering intra jugular injections on the first day after implantation of the i.c. tumour cells and again on days 9, 17, 20, & 25. The volume of p97ADR injected was measured so as to deliver 0.55 mg of ADR per Kg body weight per dose. Regular injections therefore were typically between 200 and 300 μl. The groups studied were, (1) treated with sterile PBS, (2) treated with p97 ADR conjugate containing a total of 2.75 mg/Kp ADR (i.e. from 50-300 mg p97/kg), (3) treated with a five fold concentrated solution of p97-ADR conjugate containing a total of 13.75 mg/Kg ADR (from 230 to 1500 mg p97/kg). p97-ADR was prepared according to the protocols set out above, as modified below:

A. Procedure for Cross-linking of p97 to ADR.

Preparing SATA Derivatives

Materials: Buffer 1: PBS, p97 at approx.1.5 mg/mL.

Reaction: Dissolve 10 mg of SATA in 1.0 mL DMSO, immediately before use.

Combine 0.5 mL of p9⁷ with 50 μL of SATA in 8 eppendorf tubes, and allow to react at room temperature for 60 min.

Purification:

Excelulose GF-5 desalting columns used for separation. Equilibrate 4 columns with 10 mL of Buffer 1. Apply 1 mL of reaction mixture to each column. Collect fractions at 1 min. intervals. Monitor A₂₈₀ of fractions; and pool fractions containing p97 resulting in a total of 4 mL.

Activation of ADR

Materials: ADR in 75% DMSO at 2.5 mg/mL, Sulfo-SMCC in DMSO: 14.6 mg in 10.22 μL W 33 mM. Reaction: Combine 75 μL of ADR with 75 μL of prepared sulfo-SMCC in 8 eppendorf tubes. Incubate for 3.5 hours at room temperature.

Deacyation of p97-SATA

Materials. Deacelylation solution: 0.35 g of Hydroxylamine-HCl and 0.073 g of EDTA in 8 mL of 62.5 mM Sodium, Phosphate, pH 7.5 buffer. Readjust the pH to 7.5 with NaOH and bring to a final volume of 10 mL. Final concentration of this buffer is 50 mM Sodium Phosphate. 25 mM EDTA, 0.5 M Hydroxylamine, pH 7.5.

Reaction: Combine 0.2 mL of the pooled p97-SATA fraction with 20 μL of deacelylation solution in 20 eppendorf tubes, and allow to react for 2 hours at room temperature.

Conjugation of p97 SH with ADR-SMCC

Reaction: Mix 0.22 mL of deacylated p97 with 60 μL of ADR-SMCC mixture in eppendorf tubes. Incubate at 4° C. overnight.

Purification: Use Excellulose GF-5 desalting columns for separation. Equilibrate 4 columns with 10 mL of Buffer 1. Apply 1-1.5 mL of reaction mixture to each colum. Collect all the fractions with red colour. Pool fractions containing p97 resulting in a total of 6-8 mL.

Test fur Conjugation: ELISA test to measure concentration of p97 in the conjugated fraction. Determine the LD50 on sensitive cells.

The Synapse Technologies Inc. batch of p97 used in both intra cranial studies was B06.00. Analysis by absorbance at 280 nm and 486 nm via spectrophotometer gave an estimate of 0.763 μg/mL p97 protein. This would give a ratio of 2.6 ADR per p97. The value obtained with HPLC gave an estimate of an average of 1.5 ADR per p97.

Mouse survival was recorded. Mice were euthanized according to approved protocols upon identification of morbidity. Signs of morbidity that were used as an end point for the intracranial model were behavioral changes such as decreased activity, loss of appetite or water intake, and lack of grooming and if any of the animals lost 15% of their body weight at the start of the study.

Results are set out in FIG. 9. Summary statistics are in the table below.

Summary Statistics

| PBS | 1 × (p97ADR) | 5 × (p97ADR) |
|---|---|---|
| % Inc in Median Survival over | 35% | 35% |
| PBS = | | |
| % Inc in Mean Survival over | 28% | 30% |
| PBS = | | |

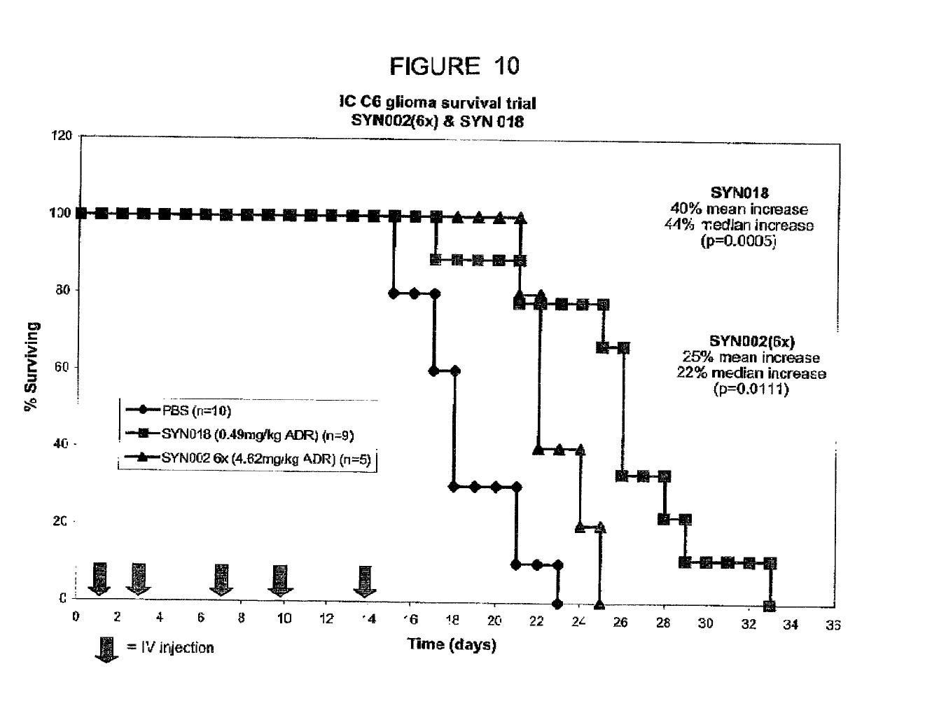

In a modified repeat experiment, results shown in FIG. 10, the identical protocol was employed to implant the i.e. tumours. Treatment of the mice was somewhat modified, as follows:

In FIG. 10 efficacy of p97-ADR against C6 growth was assesssed by administering tail vein injections on the first day after implantation of the i.c. tumour cells, and again on days 3, 7, 10 and 14. The volume of injection was 60 μl per 10 g body weight (i.e. a 25 g mouse was 150 μl). The groups studied were treated at each dose with (1) sterile PBS, (2) p97 protein alone (5×=143.8 mg/kg of p97 protein; 10×=273.8 mg/kg); (3) p97-ADR conjugate prepared as follows: SYN018 0.49 mg/kg ADR and SYN002 6× (4.62 μg/kg ADR). SYN018 and SYN002 were synthesized according to the general procedure set out above (SMCC/SATA).

Both the experiments shown in FIGS. 9 and 10 demonstrate that treatment with p97-ADR provides a statistically significant improvement in survival of mice bearing intracranial tumours. In FIG. 9, the Kaplan Meier survival curves, when analyzed using a Mantel-Haenszel log rank test, showed significantly increased survival with 1×p97ADR treatment (p<0.0l). Statistically significant differences are not observed between the low dose/low concentration formulation (2.75 mg/kg ADR); and the higher dose/higher concentration formulation (13.75 mg/kg ADR). This study shows increased mean survival duration greater than 25% as required by the US National Cancer Institute to indicate primary therapeutic potential.

In FIG. 10, results again show that p97-ADR treatment leads to statistically significant improvements in survival of i.e. tumour bearing mice.

### Example 12

### Improved Survival with Treatment with p97-ADR

The human disease model employed was intracranially implanted human ZR-75-1 mammary tumour cells (ATCC CRL-1500) xenografted in athymic nude mice (female, NCr-nu Taconine farms Inc). ZR-75-1 cells were cultured and implanted in mice as follows: A cell suspension was prepared from the tissue cultured line. Cells are mechanically scraped from the plates of flasks and washed twice by certification at 1000 rpm in RPMI 1640 or Hank's balanced salt solution (HBSS) without the serum. The cells are then resuspended in serum free RPNH 1640 or HBSS to give a concentration of 2×10⁵ viable cells per 0.025 mL per mL. Mice are placed under anesthesia with sodium pentobarbitol or chloral hydrate and the cells are implanted in the right cerebral hemisphere with a cc syringe with a 26-gauge needle filled with a sleeve that allows only a 3 mm penetration.

The IC implant size for this trial was 1×10⁶ ZR-75-1 cells per animal. The tumour cells were from cell culture p97-ADR batch B06.00, which was synthesized and prepared as set out in example 11, above.

Treatments consisted of PBS, 1×p97-ADR (5.5 mg/kg ADR), 5×p97-ADR (27.5 mg/Kg ADR), and free ADR (25 mg/kg) as a reference compound. The treatment schedule for the p97-ADR conjugate was two courses daily for five days beginning on day three and again on day ten. The treatment schedule for ADR was daily for five days beginning on day three for one course only.

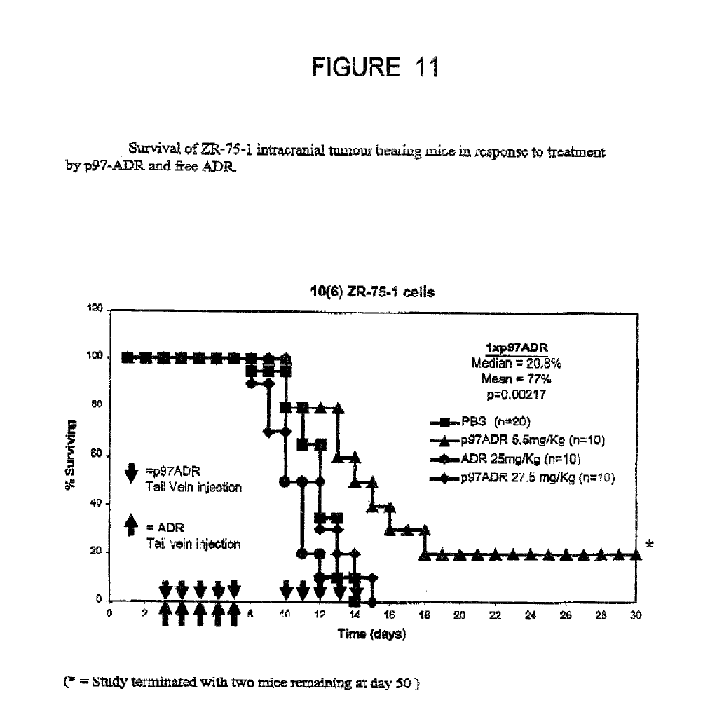

Results are set forth in FIG. 11 and the chart below.

Summary Statistics

| PBS | 1 × p97ADR | 5 × p97ADR | ADR |
|---|---|---|---|
| % Inc in Median Survival over | +20.8% | −8.3% | −12.5% |
| PBS = | | | |
| % Inc in Mean Survival over | +77% | −5.1% | −7.6% |
| PBS = | | | |

Treatment with p97-ADR results in a statistically significant improvement in survival of mice bearing ZR-75-1 intracranial tumours compared to PBS control and free ADR reference standards. Kaplan-Meier survival curves, when analyzed using a Mantel-Haenszel log rank test, showed significantly increased survival with 1×p97ADR treatment (p<0.0l). These results show increased mean survival duration greater than 25% (as required by the NCI to indicate primary therapeutic potential). Free ADR is not known to be effective against intracranial tumours, so these results demonstrate for the first time the clinically relevant discovery that conjugation of ADR to p97 dramatically enhances treatment of neurological tumours by chemotherapeutic agents in human disease models.

It is also discovered that p97-ADR conjugate is statistically not more effective in the high dose/high concentration formulation of 27.5 mg/Kg than in the low dose/low concentration formulation. One of the many possible explanations for this effect, none of which are excluded by this explanation, is that the p97 protein may be denatured or otherwise compromised in the high concentration formulation, thus preventing higher efficacy with the higher dose formulation. Protein stabilizers or other preparation techniques may be employed to obtain higher dose formulations that are therapeutically more effective.

All publications, patents and patent applications mentioned in this specification are herein incorporated by reference into the specification in their entirety for all purposes. Although the invention has been described with reference to preferred embodiments and examples thereof, the scope of the present invention is not limited only to those described embodiments. As will be apparent to persons skilled in the art, modifications and adaptations to the above-described invention can be made without departing from the spirit and scope of the invention, which is defined and circumscribed by the appended claims.

TABLE 1

| | AMOUNT OF | Amount of taxol extracted |
|---|---|---|
| | TAXOL | Brain |
| Treatment method | INJECTED | 1 hr |
| P97-TAXOL (synthesized | 156 μg | 25 ng |
| according to example | | |
| 5b(I), set out above | | |
| TAXOL | 500 ug | not detectable |

TABLE 2

| | INJECTED | Amount of ADR extracted (ng) |
|---|---|---|
| Compound | (MG) | Brain + tumour |
| FREE ADR | 2O μg | 3 +/− 2.1 ng |
| P97 ADR (APPROX. 1:8 | 20 μg | 130 +/− 4.3 ng |
| MOL:MOL) | | |

### REFERENCES

Fishman J B, Rubin J B, Handrahan J V, Connor J R, Fine R E. Receptor-mediated transcytosis of transferrin across the blood-brain barrier. J. Neurosci. Res. 18:299-304, 1987.

Banks W A, Shally A V, Barrera C M, Fasold M B, Durham D A, Csernus V J, Groot K, Kastin A J. Permability of murine blood-brain barrier to some octapeptide analogs of somatostain. Proc. Natl. Acad. Sc. USA 87:6762-6766, 1990.

Jefferies W A, Brandon M R, Hunt S V, Williams A F, Gatter K C, Mason D Y. Transferrin receptor on endothelium of brain capillaries. Nature 312:162-163, 1984

Triguero D, Buciak J, Pardridge W M. Capillary depletion method for quantification of blood-brain barrier transport of circulating peptides and plasma proteins. J. Neurochem. 54:1882-1888, 1990.

Frieden P M, Walus L R, Musso G F, Taylor M A, Malfroy B, Starzyk R M. Anti-tranferrin receptor antibody and antibody-drug conjugates cross the blood-brain barrier. Proc. Natl. Acd. Sci. USA 88;4771-4775, 1991

Rothenberger S, Food M R, Gabathuler R, Kennard M L, Yamada T, Yasuhara O, McGeer P L, Jefferies W A. Coincident expression and distibution of melanotransferrin and transferrin receptor in human brain capillary endothelium. Brain Res. 712:117-121, 1996.

Jefferies W A. Food M R, Gabathuler R, Rothenberger S, Yamada T, Yasuhara O, McGeer P L. Reactive microglia specifically associated with amyloid plaques in Alzheimer's disease brain tissue express melanotransferrin. Brain Res. 712: 122-126, 1996.

Fishman J B, Rubin J B, Handrahan J V, Connor J R, Fine R E. Receptor-mediated transcytosis of transferrin across the blood-brain barrier. J. Neurosci. Res. 18:299-304, 1987.

Duffy K R, Pardridge W M, Blood-brain barrier transeytosis of insulin in developing rabbits Brain Res 420:32-38, 1987

Nakano M, Matsui H, Miaki K, Yamagani T, Tsuji H. Postlaminectomy adhesion of the Cauda Equina. Spine 22:1105-1114, 1997

What is claimed is:

1. A pharmaceutical composition, said pharmaceutical composition comprising:
a chemotherapeutic agent conjugated to p97; and
a pharmaceutically acceptable carrier therefor.

2. The composition of claim 1, wherein the chemotherapeutic agent is selected from the group consisting of adriamycin, cisplatin, paclitaxel, camptothecin, 5-fluorouracil, and analogs thereof.

3. The composition of claim 2, comprising p97-adriamycin.

4. The composition of claim 2, wherein said chemotherapeutic agent comprises at least two different chemotherapeutic agents.

5. The composition of claim 2, wherein said chemotherapeutic agent comprises a cardiotoxic agent.

6. The composition of claim 2, wherein conjugation of said chemotherapeutic agent to p97 involves a linker comprising an organic moiety, selected from the group consisting of an alkyl, acryl or amino acid backbone.

7. The composition of claim 6, wherein said organic moiety comprises an amide, ether, ester, hydrazone, sulphide or disulphide linkage or any combination thereof.

8. The composition of claim 6, wherein said conjugation is stable at physiological pH.

9. The composition of claim 8, wherein said conjugation is unstable at intracellular pH.

10. The composition of claim 2, wherein said conjugation is by a sulfhydryl linker.

11. The composition of claim 2, wherein the molar ratio of said chemotherapeutic agent to p97 is at least 2:1.

12. The composition of claim 2, wherein the molar ratio of said chemotherapeutic agent to p97 is at least 7:1.

13. The composition of claim 2, wherein the molar ratio of chemotherapeutic agent to p97 is at least 10:1.

14. The composition of claim 10, 11 or 12, wherein said chemotherapeutic agent is adriamycin.

15. The composition of claim 1, where the chemotherapeutic, agent is selected from the group consisting of alkylating agents, antimetabolites, natural products (such as vinca alkaloids, epidophyllotoxins, antibiotics, enzymes and biological response modifiers), topoisomerase inhibitors, microtubule inhibitors, spindle poisons, hormones and antagonists, and miscellaneous agents such as platinum coordination complexes, anthracendiones, substituted ureas, and analogs thereof.

16. A pharmaceutical compositional said pharmaceutical composition comprising:
a chemotherapeutic agent conjugated to p97 comprising a dosage unit of from about 0.02 to about 2000 mg/kg of p97, substantially all of which p97 is conjugated to said chemotherapeutic agent; and
a pharmaceutically acceptable carrier therefor.

17. The composition of claim 16, wherein said chemotherapeutic agent is selected from the group consisting of adriamycin, cisplatin, paclitaxel, or an analog thereof.

18. The composition of claim 16, wherein said dosage unit comprises from about 0.1 to about 100 mg/kg of said chemotherapeutic agent.

19. The composition of claim 16, wherein said dosage unit comprises from about 0.1 to about 10 mg/kg of p97.

20. A method for increasing delivery of a chemotherapeutic agent to a neoplasia localized in or around the brain, said method comprising administering a p97-chemotherapeutic agent to an animal having a neoplasia in or around the brain, wherein the amount of chemotherapeutic agent delivered as part of the p97-chemotherapeutic agent to said neoplasia is increased relative to delivery of the chemotherapeutic agent when said chemotherapeutic agent is not conjugated to p97 and administered at an equivalent dose.

21. A method according to claim 20, said method comprising:
a) conjugating a chemotherapeutic agent to p97 to generate a p97-chemotherapeutic agent; and
b) administering said p97-chemotherapeutic agent to an animal having a neoplasia in or around the brain, wherein the amount of chemotherapeutic agent delivered as part of the p97-chemotherapeutic agent to said neoplasia is increased relative to delivery of the chemotherapeutic agent when said chemotherapeutic agent is not conjugated to p97 and administered at an equivalent dose.

22. A method for targeting a chemotherapeutic agent to a neoplasia localized in or around the brain, said method comprising administering a p97-chemotherapeutic agent to an animal having a neoplasia localized in or around the brain, wherein said patient experiences increased delivery of said chemotherapeutic agent to said neoplasia compared to when the chemotherapeutic agent is not conjugated to p97 and is administered at an equivalent dose.

23. A method according to claim 22, said method comprising:
a) conjugating a chemotherapeutic agent to p97 to generate a p97-chemotherapeutic agent, and
b) administering the p97-chemotherapeutic agent to an animal having a neoplasia localized in or around the brain, wherein said patient experiences increased delivery of said chemotherapeutic agent to said neoplasia compared to when the chemotherapeutic agent is not conjugated to p97 and is administered at an equivalent dose.

24. A method of treating a neoplasia localized in or around the brain comprising administering to a subject in need thereof a pharmaceutically effective amount of a p97-chemotherapeutic agent.

25. The method of claim 24, wherein said p97-chemotherapeutic agent is p97-adriamycin.

26. The method of claim 25, wherein the molar ratio of adriamycin to p97 is at least 2:1.

27. The method of claim 25, wherein the molar ratio of adriamycin to p97 is at least7:1.

28. The method of claim 25, wherein the molar ratio of adriamycin to p97 is at least 10:1.

29. A p97-chemotherapeutic agent conjugate selected from the group consisting of:

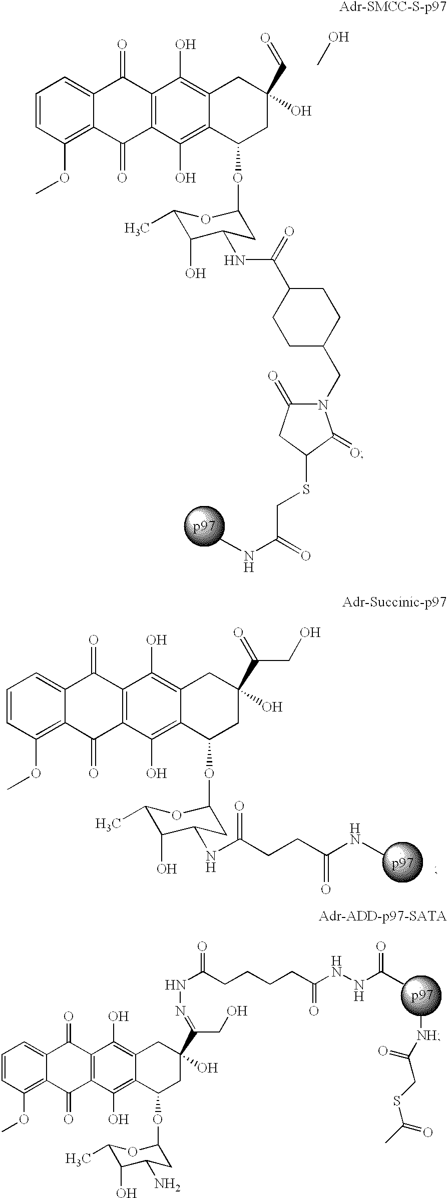

30. A modified adriamycin molecule selected from the group consisting of:

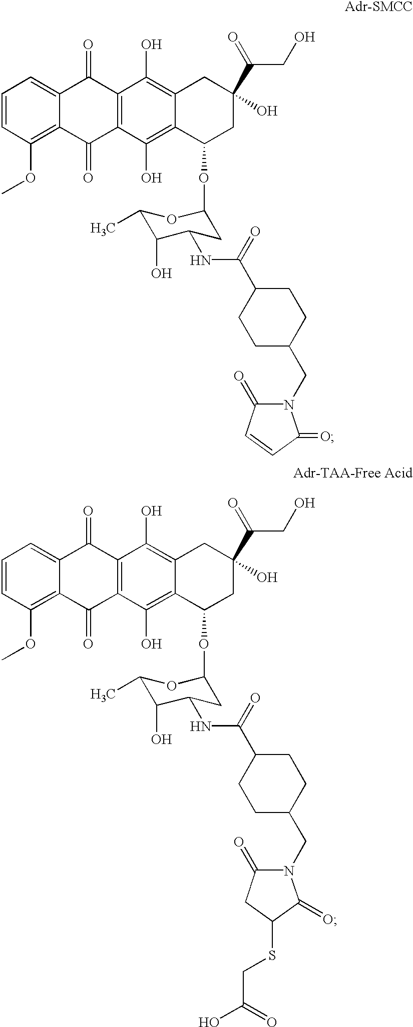

31. A method of preparing a p97-adriamycin conjugate comprising the steps of:
dissolving adriamycin in an inert solvent, and adding an organic base, preferably triethylamine;
adding a solution of SMCC in an inert solvent,
adding mercapto acetic acid;
adding a coupling reagent;
adding the solution of adriamycin, base, SMCC, mercaptoacetic acid and coupling reagent slowly to a solution of p97 and reacting under conditions to provide adriamycin-p97 conjugates; and
purifying the adriamycin-p97 conjugates.

32. The method according to claim 31, wherein the inert solvent is DMF.

33. The method according to claim 31, wherein the organic base is a trialkylamine.

34. The method according to claim 31, wherein the coupling reagent is O-benzotriazol-1-yl-N,N,N′,N′-tetramethyluronium tetrafluoroborate (TBTU).

35. A method of detecting or diagnosing a cancer of the brain comprising administering an effective amount of a composition comprising a radioimaging agent conjugated to p97 to an animal in need thereof.

36. A method according to claim 35 wherein the radioimaging agent is technetium-99M.
